(19) United States
(12) Patent Application Publication
(10) Pub. No.: US 2015/0352107 A1
(43) Pub. Date: Dec. 10, 2015

(54) ALPHA7 NEURONAL NICOTINIC RECEPTOR LIGAND AND ANTIPSYCHOTIC COMPOSITIONS

(71) Applicant: AbbVie Inc., North Chicago, IL (US)

(72) Inventors: Kathy L. Kohlhaas, Lake Zurich, IL (US); Lynne E. Rueter, Round Lake Beach, IL (US); R. Scott Bitner, Pleasant Prairie, WI (US)

(21) Appl. No.: 14/830,588

(22) Filed: Aug. 19, 2015

Related U.S. Application Data
(63) Continuation of application No. 11/370,419, filed on Mar. 8, 2006.
(60) Provisional application No. 60/663,184, filed on Mar. 18, 2005.

(51) Int. Cl. A61K 31/501; A61K 31/439; A61K 31/519; A61K 31/451; A61K 31/5513

(52) U.S. Cl. CPC A61K 31/501; A61K 31/451; A61K 31/5513; A61K 31/519; A61K 31/439

(57) ABSTRACT

The present invention relates to a composition comprising an antipsychotic and an α7 nicotinic acetylcholine receptor ligand, a method of using the same, and a related article of manufacture.

This application claims the benefit of U.S. Patent Application No. 60/663,184, filed on Mar. 18, 2005, which is hereby incorporated by reference.

### BACKGROUND OF THE INVENTION

1. Technical Field

The present invention relates to a composition comprising an antipsychotic and an α7 nicotinic acetylcholine receptor ligand, a method of using the same, and a related article of manufacture.

2. Description of Related Technology

Psychotic conditions such as schizophrenia and related disorders, for example schizoaffective disorder, are complex and heterogeneous diseases of uncertain etiology. With a worldwide prevalence of approximately one percent to two percent of the population, schizophrenia has serious social and economic consequences.

Schizophrenia itself is characterized by fundamental distortions in realms of thinking and perception, cognition and the experience of emotions. With a typical onset in late adolescence or early adulthood, it is a chronic lifelong illness with periods of frank psychotic features alternating with periods of residual symptoms and incomplete social recovery. Schizophrenia requires medical intervention in virtually all cases. Approximately 60% to 70% of schizophrenic patients-never marry and the unemployment rate among schizophrenic patients is greater than 70%. Such statistics suggest that schizophrenic patients do not adequately function in society.

Symptoms of schizophrenia are subdivided into three major clusters: positive, negative, and cognitive. Positive (psychotic) symptoms, consist of delusions (false beliefs that cannot be corrected by reason), hallucinations (usually nonexistent voices), disorganized speech, and grossly disorganized behavior. Negative symptoms are described as affective flattening, alogia (speechlessness caused by mental confusion), avolition (lack of motivation to pursue a goal), and anhedonia (inability to experience pleasure). Cognitive deficits include impairments of working memory, attention, verbal reproduction, and executive function. Furthermore, a variety of associative features and mental disorders include poor insight, depersonalization, derealization, depression, anxiety, and substance abuse disorders. Finally, schizophrenia patients have a markedly increased risk of suicide rate with 20% to 40% attempting suicide at least once in their lifetime, and 10% of patients successively committing suicide. (DSM-IV Diagnostic and Statistical Manual of Mental Disorders, 4^{th} edition, American Psychiatric Assoc., Washington, D.C., 2000).

The current standard of treatment for schizophrenia is the atypical antipsychotics, although there is still significant use of typical antipsychotics throughout the world. Typical antipsychotic drugs (phenothiazines, butryophenones, and thioxanthenes), which are also referred to as conventional, standard, classical, or first generation antipsychotic drugs, have until recently, been the core treatment of schizophrenia.

A limitation of treatment with the typical antipsychotics is the induction of extrapyramidal side effects (EPS). EPS include Parkinsonism, dystonia, akathisia and neuroleptic malignant syndrome as well as the irreversible movement disorder called tardive dyskinesia. Severe akathisia can cause patients to feel anxious or irritable and can result in aggressive or suicidal acts. The most troublesome neurological side effect, tardive dyskinesia, can be irreversible, the risk of which has been a major rationale for preference of atypical over typical drugs. The occurrence of EPS is dose dependent and occurs in up to 60% of patients treated with typical antipsychotics. In practice, clinicians titrate the dose for each patient in order to achieve the greatest efficacy with a manageable level of side effects. (Kinon et al, CNS Drugs, 2004, 18:597-616; Tarsy et al, CNS Drugs, 2002, 16:23-45; Kulisevsky and Otermin, Neurologia, 2003, 18:262-268). Thus, potential efficacy of the antipsychotic agent is limited by the narrow therapeutic window. Atypical antispsychotics typically are drugs that have at least equal antispychotic efficacy and produce fewer discomforting acute and long-term adverse effects. These medications are generally accepted to be effective in controlling positive symptoms although their efficacy in other aspects of the disorder (e.g. control of negative symptoms and cognitive deficits) is controversial. Some of the newer atypical antipsychotics have a reduced liability, i.e. a greater therapeutic window in which to titrate efficacy, compared to typical antipsychotics. For example, atypical antipsychotics such as clozapine, risperidone, olanzapine, and sertindole have a decreased risk of EPS induction as compared to the typical antipsychotics; however, such atypical antipsychotics can still induce EPS in greater than 30% of patients. Clozapine is an exception in that it produces few extrapyramidal side effects; however, this atypical neuroleptic is known to produce blood dyscrasias that also limit its use.

In addition to EPS, currently available antipsychotics produce other side effects that limit their usefulness, the physician's ability to titrate to the optimal dose necessary to control the symptom clusters of the disorder, or both. These include secondary negative symptoms such as anhedonia, cognitive impairment, weight gain, metabolic syndrome, and diabetes.

There is some suggestion that atypical antipsychotics have increased efficacy in treating negative and cognitive symptoms. However, only clozapine is commonly accepted to have efficacy against these other symptom clusters. Moreover, clozapine is only approved for otherwise treatment-refractory patients due to the risk of agranulocytosis. (Practice Guidelines for the Treatment of Psychiatric Disorders Compendium 2002, American Psychiatric Assoc., Washington, D.C., 2002; Kapur and Remington, Ann. Rev. Med, 2001, 52:503-517).

Various adjunctive treatments have been with antipsychotic medications. However, as noted below, the purpose of the adjunctive therapy differs.

Antiepileptics including vaiproate, benzodiazepines, L-dopa, and quetiapine, have been suggested or demonstrated to improve positive symptoms with little mentioned effect on EPS.

Antidepressants (for example fluvoxamine, mirtazapine, reboxetine, nefazadone), glycine, 5-HT1A agonists, and glucose, have been suggested or demonstrated to improve negative, cognitive, or depressive symptoms with little mentioned effect on EPS.

Fluoxetine has been shown to exacerbate EPS when used as adjunctive therapy for negative/depressive symptoms.

Anticholinergics, beta blockers, antioxidants, benzodiazepines, L-dopa, and histamine H₂ antagonists such as famotidine, amantadine, metformin, topiramate, and orlistat, have been suggested or demonstrated to reduce antipsychotic-induced side effects including EPS and weight gain.

Nizatidine has been shown to exacerbate EPS when used to control weight gain.

The adverse effects associated with the antipsychotics can lead to treatment noncompliance or treatment termination and, as such, increase the rate of relapse and rehospitalization during the course of the chronic illness. (Practice Guidelines for the Treatment of Psychiatric Disorders Compendium 2002, American Psychiatric Assoc., Washington, D.C., 2002; Kapur and Remington, Ann. Rev. Med, 2001, 52:503-517). As a result of the limited efficacy and the side effects, lack of patient compliance in taking medications is a serious problem in the treatment of schizophrenia. More than 40% of schizophrenic patients fail to take their medication as prescribed.

With the exception of tardive dyskinesia, EPS can be resolved by discontinuing treatment with the medication. However, discontinuing treatment puts the patient at risk of schizophrenia symptom relapse.

Accordingly, successful treatment using currently available antipsychotics is limited by the wide range of side effects associated with their use, albeit to differing degrees. CNS diseases such as psychotic disorders are an unmet medical need, and the methods and possibilities for treatments of such indications are insufficient. In light of the significance of psychotic disorders and the limitations in their treatment, it would be beneficial to identify new methods of treating such psychotic disorders, particularly in a manner that reduces the risk of EPS.

### SUMMARY OF THE INVENTION

The present invention relates to a composition for treatment of individuals with psychotic and related disorders, which involves a combination of an antipsychotic drug with a nicotinic acetylcholine receptor (nAChR) ligand, particularly α7 subtype receptor ligand. The present invention provides a synergistic combination of an antipsychotic drug with a nicotinic acetylcholine receptor ligand, for example an α7 neuronal nicotinic receptor agonist or an allosteric modulator. The present invention further provides for the treatment or prevention of central nervous system disorders, including psychotic disorders, especially in humans. Such combination reduces a patient's exposure to EPS and can provide a beneficial alternative to current treatments.

In one embodiment, the present invention relates to a composition comprising (i) an antipsychotic drug; and (ii) a neuronal nicotinic receptor subtype α7 receptor ligand, in admixture with at least one pharmaceutically acceptable excipient. The present invention is most beneficial wherein the amounts of (i) and (ii) are together effective in treating a psychotic disorder, particularly with less EPS. However, a composition wherein (i) and (ii) are each present in an effective amount also is contemplated. The antipsychotic drug can be a neuroleptic dopamine receptor antagonist or any other typical or atypical antipsychotic useful for treatment of schizophrenia or other psychotic related disorders.

In another embodiment, the present invention relates to a method for treating or preventing a psychotic condition in a patient. In the method, the steps include, but are not limited to, (i) administering an antipsychotic drug to a patient; and (ii) administering a neuronal nicotinic receptor subtype α7 receptor ligand to a patient to treat or prevent a psychotic condition.

Yet another embodiment relates to an article of manufacture, having (i) a first pharmaceutical dosage form with at least one antipsychotic; (ii) a second pharmaceutical dosage form with at least one neuronal nicotinic acetylcholine subtype α7 receptor ligand; and wherein the article contains first and second pharmaceutical dosage forms.

The embodiments of the present invention, how to prepare them, and how to use them are further described herein.

### BRIEF DESCRIPTION OF THE DRAWINGS

FIGS. 1A, 1B, and 1C graphically represent the effects of clinically used antipsychotic drugs such as risperidone, haloperidol, and clozapine, respectively, to enhance the effect in a prepulse inhibition study in DBA2 mice. These compounds are representative of the various types of antipsychotic drugs used in clinical practice.

FIG. 2 graphically represents the effect of Compound 1, 5-(6-[(3R)-1-azabicyclo[2.2.2]oct-3-yloxy]pyridazin-3-yl-1H-indole, in boosting the effect of a sub efficacious dose of risperidone, an atypical antipsychotic.

FIG. 3 graphically represents the effect of Compound 1 on a side effect associated with risperidone, such as drug-induced catalepsy.

FIG. 4 graphically represents the effect of Compound 1 in boosting the effect of a sub efficacious dose of haloperidol, a typical antipsychotic.

FIG. 5 graphically represents that Compound 1 does not interfere with the efficacy of haloperidol.

FIG. 6 graphically represents the effects of α7 neuronal nicotinic agonist Compound 2, 2-(6-phenylpyridazin-3-yl)octahydropyrrolo[3,4-c]pyrrole, in a prepulse inhibition study in DBA2 mice.

FIG. 7 graphically represents that Compound 2 potentiates the efficacy of risperidone.

FIG. 8 graphically represents the effects of α7 neuronal nicotinic agonist Compound 3, N-(3R)-1-azabicyclo[2,2,2]oct-3-yl-4-chlorobenzamide fumarate, in a prepulse inhibition study in DBA2 mice.

FIG. 9 graphically represents the effect of Compound 3 in boosting the effect of a sub efficacious dose of risperidone.

FIG. 10 graphically represents the effect of Compound 1 on a side effect associated with haloperidol, such as drug-induced catalepsy.

FIG. 11 graphically represents the effect of Compound 2 on a side effect associated with risperidone, such as drug-induced catalepsy.

FIG. 12 graphically represents the effect of Compound 3 on a side effect associated with risperidone, such as drug-induced catalepsy.

### DETAILED DESCRIPTION OF THE INVENTION

### Antipsychotic Drugs

Typical, or classical, antipsychotics and atypical antipsychotics are well known to those skilled in the art.

Typical antipsychotics demonstrate antagonism at the dopamine D2 receptors. Typical antipsychotics generally are classified into three groups according to their potency. For example, typical antipsychotics include high affinity agents, such as haloperidol and fluphenazine; intermediate potency agents, such as loxapine; and low potency agents, such as chlorpromazine. Typical antipsychotics are associated with efficacy against positive symptoms but with significant incidence of side effects including EPS and sedation.

Atypical antipsychotics demonstrate a high level of affinity for the 5HT₂ receptor and functions as an antagonist of serotonin at that receptor. While the exact mechanism by which these compounds exert their antipsychotic effect is still under review, it is believed that at least part of their efficacy stems from their ability to modulate serotonergic transmission within the CNS. While atypical antipsychotics often have affinity for dopaminergic receptors within the CNS, they are much less potent dopaminergic antagonists than classical antipsychotics, such as chlorpromazine, haloperidol, and others. For a detailed discussion of these compounds and their mechanism of action, the readers attention is directed to Blin, Comparative Review of New Antipsychotics, Can J Psychiatry, Vol 44, 235-242 Apr. 1999. In addition to their differing mechanism of action, atypical antispychotics can be differentiated from classical antipsychotics based upon their side effect profile. Atypical antipsychotics are associated with a significantly reduced incidence of acute extrapyramidal symptoms, especially dystonias, when compared to a typical antipsychotic such as haloperidol. (Beasley, et al., Neuropsychopharmacology, 14(2), 111-123, (1996); Ananth J, et al., Curr. Pharm. Des. 10(18):2219-29 (2004)).

Typical antipsychotic agents can include compounds that are D₂ antagonists, for example, phenthiazines, butryrophenones, and thiozanthenes. Examples of such classes of compounds include, but are not limited to, fluphenazine, chlorpromazine, haloperidol, and loxapine.

Atypical antipsychotic agents can include compounds that are mixed antagonists that usually, but are not limited to, demonstrate D₂ and 5-HT1A antagonism. Examples include clozapine, risperidone, olanzapine, quetiapine, ziprasidone, and arpiprazole.

Adjunctive antipsychotic agents can include compounds that are antiepileptics, antidepressants, or anticholinergics. Examples of such classes of compounds include, but are not limited to, beta blockers, antioxidants, benzodiazepines, L-dopa, H₂ antagonists, and 5HT1A agonists.

Any other compound having a pharmacological profile or clinical benefit analogous to the compounds described above or other compounds emerging via targeting subtypes or subunits of receptors, ion channels, enzymes, or other mechanisms, should also be considered to be encompassed by the term antipsychotic even if that compound is discovered after the filing of this application.

Examples of suitable typical antipsychotics include, but are not limited to the following compounds, below.

Haloperidol (Haldol), 4-(4-chlorophenyl)-1-[4-(4-fluorophenyl)-4-oxobutyl]-4 piperidinyl, is available in oral (solution, tablets) or in a parenteral form from Ortho McNeil Pharmaceuticals. Haloperidol decanoate, which is administered intramuscularly as a depot preparation, is an alternative for long-term therapy.

Chlorpromazine (Thorazine, Largactil), 10-(3-dimethylaminopropyl)-2-chlorphenothiazine, is available in oral or in parenteral form from GlaxoSmithKline and others.

Fluphenazine (Modecate, Permitil, Prolixin), 4-[3-[2-(trifluoromethyl)phenothiazin-10H-yl]propyl]-1-piperazineethanol, is available in oral or in parenteral form from Boehringer Ingeheim and others. Fluphenazine deconoate, which is administered intramuscularly as a depot preparation, is an alternative for long-term therapy.

Examples of suitable atypical antipsychotics include, but are not limited to, the following compounds, below.

Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, and its use in the treatment of psychotic diseases are described in U.S. Pat. No. 4,804,663. Risperidone is available commercially from Janssen. A detailed discussion of risperidone, its dosing schedule, potential side effects, and other information, may be found in AHFS, Drug Information 2000, page 2142, which is published by the American Society of Hospital Pharmacists (editor-McEvoy).

Olanzapine, 2-methyl-4-(4-methyl-1-piperazinyl)-10H-thieno[2,3-b][1,5]benzodiazepine, is a known compound and is described in U.S. Pat. No. 5,229,382 as being useful for the treatment of schizophrenia, schizophreniform disorder, acute mania, mild anxiety states, and psychosis. U.S. Pat. No. 5,229,382. Olanzapine is available commercially from Eli Lilly. A detailed discussion of olanzapine, its dosing schedule, potential side effects, etc., may be found in AHFS, Drug Information 2000, page 2135, which is published by the American Society of Hospital Pharmacists (editor-McEvoy).

Clozapine, 8-chloro-11-(4-methyl-1-piperazinyl)-5H-dibenzo[b,e][1,4]diazepine, is described in U.S. Pat. No. 3,539,573. Clinical efficacy in the treatment of schizophrenia is described by Hanes et al, Psychopharmacol. Bull., 24, 62 (1988). Clozapine is available commercially from Novartis. A detailed discussion of clozapine, its dosing schedule, potential side effects, etc., may be found in AHFS, Drug Information 2000, page 2125, which is published by the American Society of Hospital Pharmacists (editor-McEvoy).

Quetiapine, 5-[2-(4-dibenzo[b,f][1,4]thiazepin-11-yl-1-piperazinyl)ethoxy]ethanol, and its activity in assays which demonstrate utility in the treatment of schizophrenia are described in U.S. Pat. No. 4,879,288. Quetiapine is typically administered as its (E)-2-butenedioate (2:1) salt. It is available commercially from Astra Zeneca. A detailed discussion of quetiapine, its dosing schedule, potential side effects, and other aspects of the treatment, may be found in AHFS, Drug Information 2000, page 2142, which is published by the Americal Society of Hospital Pharmacists (editor-McEvoy).

Ziprasidone, 5-[2-[4-(1,2-benzoisothiazol-3-yl)-1-piperazinyl]ethyl]-6-chloro-1,3-dihydro-2H-indol-2-one, is typically administered as the hydrochloride monohydrate. It is commercially available from Pfizer. The compound is described in U.S. Pat. Nos. 4,831,031 and 5,312,925. Its activity in assays which demonstrate utility in the treatment of schizophrenia are described in U.S. Pat. No. 4,831,031. U.S. Pat. Nos. 4,831,031 and 5,312,925.

Arpiprazole (Abilify) is an atypical antipsychotic drug that has been recently introduced for clinical use in the treatment of schizophrenia. Additional information can be obtained from Bristol-Myers Squibb. Naber, et al., in Prog Neuropsychopharmacol Biol Psychiatry. 2004 Dec. 28(8):1213-9, evaluated the antipsychotic effect of arpiprazole.

Sertindole, 1-[2-[4-[5-chloro-1-(4-fluorophenyl)-1H-indol-3-yl]-1-piperidinyl]ethyl]imidazolidin-2-one, is described in U.S. Pat. No. 4,710,500. Its use in the treatment of schizophrenia is described in U.S. Pat. Nos. 4,710,500; 5,112,838; and 5,238,945.

Zotepine, 2-{(8-chlorodibenzo[b,f]thiepine-10-yl)oxy]-N,N-dimethylethylamine, is available commercially from Knoll under the tradename Zoleptil®. It is approved for use as an antipsychotic in Japan and Germany.

Perospirone is marketed in Japan for schizophrenia by Yoshitomi. Further information regarding the compound can be obtained from Sumitomo Pharmaceutical, of Japan.

Aberrant sensory gating and altered neurotransmission mechanisms are recognized as etiological factors in schizophrenia psychopathology. One well-known aspect is that schizophrenic patients generally demonstrate the lack of an ability to gate, or sort sensory activity, appropriately. Without being limited by the theory of invention, it is believed that an α7 receptor ligand modulates sensory gating mechanisms and alter neurotransmission including neuronal firing and neuotransmitter release so as to influence the effects of antipsychotics.

Accordingly, some medicines still at early stages of development that act on the 5HT and dopamine receptors are believed to be suitable for the present invention as well. For example, EMR-62218, under investigation by Merck Pharmaceuticals, and eplivanserin (Sanofi-Synthélabo), are reported to be selective inhibitors of the 5HT2A receptor with no dopamine blockade. SSR-181507 (Sanofi-Synthélabo) is reported to be a mixed dopamine D2/5HT2A antagonist, while SB271046 (GlaxoSmithKline) is an antagonist of the 5HT6 receptor that have progressed into clinical trials. PNU-177864 (Pfizer) is reported to be a highly selective partial blocker of the dopamine D₃ receptor. SR-125047 (Sanofi-Synthélabo) is reported to be a compound that modulates a brain site called the central sigma receptor, to which haloperidol has also been shown to bind.

Rimonabant (formerly SR-141716), a blocker of the cannabinoid receptor, also may be suitable.

Neurokinin-3 antagonists, such as osanetant and talnetant (SB223412), currently are under investigation in clinical trials. Neurokinins are chemical compounds called peptides found in the substantia nigra and striatum regions of the brain. Neurokinins are involved in movement control, which are believed to be relevant to some of the side effects of neuroleptic medicines. Accordingly, it is contemplated that the combination of an α7 receptor ligand with a neurokinin-3 antagonist also will demonstrate useful adjuvant therapy in similar manner as with antipsychotic previously described.

An entirely different approach to schizophrenia is the testing of inhibitors of a brain enzyme responsible for the breakdown of polyunsaturated fatty acids in cell membranes. A compound of this type, LAX-101d (Laxdale Pharmaceuticals) has emerged into clinical trials. It is contemplated that such α7 agonists can influence neuronal activity and, in combinations with such mechanisms that influence membrane properties, could enhance effectiveness of compounds. Further information regarding how to prepare the compounds and relevant dosing information can be obtained from the respective manufacturers as clinical trials advance.

### Nicotinic Acetylcholine Subtype α7 Receptor Ligand

It has been found that the efficacy of antipsychotic drugs previously described surprisingly can be improved by combining the antipsychotic with a nicotinic acetylcholine subtype α7 receptor ligand (α7 receptor ligand). Such α7 receptor ligands are highly efficient for improving the efficacy of antipsychotic medications without exaggerating the side effect profile of such agents.

Nicotinic acetylcholine subtype α7 receptor ligands modulate the function of nicotinic acetylcholine subtype α7 receptors by altering the activity of the receptor. Suitable compounds also can be partial agonists that partially block or partially activate the α7 receptor or agonists that activate the receptor. Positive allosteric modulators are compounds that potentiate the receptor response to acetylcholine without themselves triggering receptor activation or desensitization, or either, of the receptor. Nicotinic acetylcholine subtype α7 receptor ligands suitable for the invention can include full agonists, partial agonists, or positive allosteric modulators.

One manner to characterize α7 receptor ligands is that they demonstrate Kᵢ values from about 1 nanomolar to about 10 micromolar when tested by the [³H]-MLA assay, many having a binding value (“Kᵢ MLA”) of less than 1 micromolar. [³H]-Cytisine binding values (“Kᵢ Cyt”) of compounds of the invention ranged from about 50 nanomolar to greater than 100 micromolar. The determination of preferred compounds typically considered the Kᵢ MLA value as measured by MLA assay in view of the Kᵢ Cyt value as measured by [³H]-cytisine binding, such that in the formula D=Kᵢ Cyt/Kᵢ MLA, D is at least 50. For example, preferred compounds typically exhibit greater potency at α7 receptors compared to α4β2 receptors. Although the MLA and [³H]-cytisine binding assays are well known, further details for carrying out the assays can be obtained in International Publication Nos. WO 2005/028477; WO 2005/066168; US 20050137184; US20050137204; US20050245531; WO 2005/066166; WO 2005/066167; and WO 2005/077899.

Positive allosteric modulators, at concentrations ranging from 1 nM to 10 μM, enhance responses of acetylcholine at α7 nicotinic receptors expressed endogenously in neurons or cell lines, or via expression of recombinant protein in Xenopus oocytes or in cell lines.

Accordingly, α7 receptor ligands suitable for the invention can be compounds of various chemical classes. Particularly, some examples of α7 receptor ligands suitable for the invention include, but are not limited to diazabicycloalkane derivatives, for example as described in International Publication No. WO 2005/028477; spirocyclic quinuclidinic ether derivatives, for example as described in International Publication No. WO 2005/066168; fused bicycloheterocycle substituted quinuclidine derivatives, for example as described in US Publication Nos. US20050137184; US20050137204; and US20050245531; 3-quinuclidinyl amino-substituted biaryl derivatives, for example as described in International Publication No. WO 2005/066166; 3-quinuclidinyl heteroatom-bridged biaryl derivatives, for example as described in International Publication No. WO 2005/066167; and amino-substituted tricyclic derivatives, for example as described in International Publication No. WO 2005/077899, all of which are hereby incorporated by reference in their entirety. Although it is described that the use of such α7 receptor ligands can be used in combination with antipsychotics for their cognitive benefits, the use of α7 receptor ligands for improving the efficacy of antipsychotics without exaggerating the side effect profile of such agents apparently is not contemplated.

For example, diazabicycloalkane derivatives generally can have the formula:

Z—Ar¹—Ar² (I)

or a pharmaceutically acceptable salt, ester, amide, or prodrug thereof, wherein:

Z is a diazabicyclic amine of the formula:

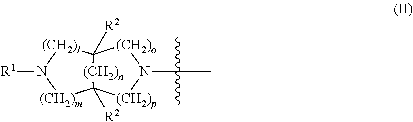

Ar¹ is a 5- or 6-membered aromatic ring of the formula (a) or (b):

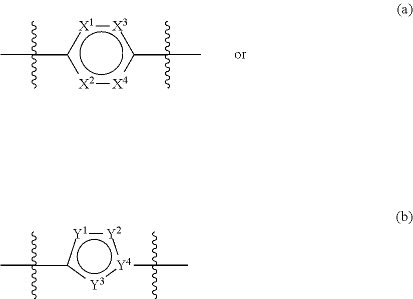

Ar² is selected from the group consisting of an unsubstituted or substituted 5- or 6-membered heteroaryl ring; unsubstituted or substituted bicyclic heteroaryl ring; 3,4-(methylenedioxy)phenyl; carbazolyl; tetrahydrocarbazolyl; naphthyl; and phenyl; wherein Ar² is substituted with 0, 1, 2, or 3 substituents selected from the group consisting of alkenyl, alkoxy, alkoxyalkoxy, alkoxyalkyl, alkoxycarbonyl, alkoxysulfonyl, alkyl, alkylcarbonyl, arylcarbonyl, alkylcarbonyloxy, alkylsulfonyl, alkylthio, alkynyl, carboxy, cyano, formyl, haloalkoxy, haloalkyl, halogen, hydroxy, hydroxyalkyl, mercapto, nitro, —NR^{A}R^{B}, (NR^{A}R^{B})alkyl, (NR^{A}R^{B})carbonyl, (NR^{A}R^{B})sulfonyl, and phenyl; provided that when Y¹ is O or S, Y² is N, Y³ is —CR³ and R³ is hydrogen, and Y⁴ is C, then Ar² is not 5-tetrazolyl;

X¹, X², X³, and X⁴ are each independently selected from the group consisting of N and —CR³, provided that R³ is not hydrogen at least in one occurrence when X¹, X², X³, and X⁴ are all —CR³;

Y¹, Y², and Y³ are each independently selected from the group consisting of N, O, S, and —CR³;

Y⁴ is selected from the group consisting of C and N, provided that when Y⁴ is C at least one of Y¹, Y², and Y³, is other than —CR³;

l, m, n, o, and p are each independently selected from the group consisting of 0, 1, or 2, provided that the sum total of l, m, n, o, and p is 3, 4, or 5, and further provided that the sum of l and o is at least 1 and the sum of m and p is at least 1;

R¹ is selected from the group consisting of hydrogen, alkenyl, alkyl alkoxycarbonyl, arylalkyl, and heteroarylalkyl;

R² at each occurrence is independently selected from the group consisting of hydrogen, alkoxycarbonyl, and alkyl;

R³ at each occurrence is independently selected from the group consisting of hydrogen and alkyl;

R^{A} and R^{B} are each independently selected from the group consisting of hydrogen, alkyl, alkylcarbonyl, alkylsulfonyl, arylcarbonyl, formyl and (NR^{C}R^{D})sulfonyl; and

R^{C} and R^{D} are each independently selected from the group consisting of hydrogen and alkyl.

One method of preparing diazabicycloalkane derivatives involves treating a commercially available 3,6-dichloropyridazines with an aryl- or heteroaryl-boronic acid, palladium(0), and a base to provide the corresponding monoarylmonochloropyridazines. The resulting monoarylmonochloropyridazines can be treated with suitable protected diazabicycle moieties and base to provide protected diazabicycle-substituted pyridazines. Such protected diazabicycle-substituted pyridazine derivatives are deprotected and alkylated using reductive amination methods well-known to those of skill in the art to provide alkylated diazabicycle-substituted pyridazines.

Another method for preparing diazabicycloalkane derivatives involves treating a suitable protected diazabicycle moiety with a dihalogenated 5- or 6-membered ring and Pd(0) with a base, such as NaOtBu or Cs₂CO₃ to provide a haloaryldiamine. The haloaryldiamine can be further treated with an aryl or heteroaryl boronic acid and Pd(0), or alternatively with an aryl or heteroaryl organostannane and Pd(0), to provide the protected biarylated diamine. Deprotection or protection and alkylation as previously described above provides suitable diazabicycloalkane derivatives. Further description for preparing diazabicycloalkane derivatives can be found in International Publication WO 2005/028477, published Mar. 31, 2005, which is hereby incorporated by reference in its entirety.

Fused bicycloheterocycle substituted quinuclidine derivatives can have the formula:

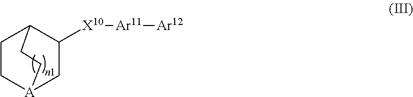

or a pharmaceutically acceptable salt, ester, amide, or prodrug thereof, wherein:

n1 is 0, 1, or 2;

A is N or N⁺—O⁻;

X¹⁰ is selected from the group consisting of O, S, and —N(R¹¹)—;
Ar¹¹ is a 6-membered aromatic ring containing 0, 1, 2, 3, or 4 nitrogen atoms, wherein Ar¹¹ is substituted with 0, 1, 2, 3, or 4 alkyl groups;

Ar¹² is a group of the formula:

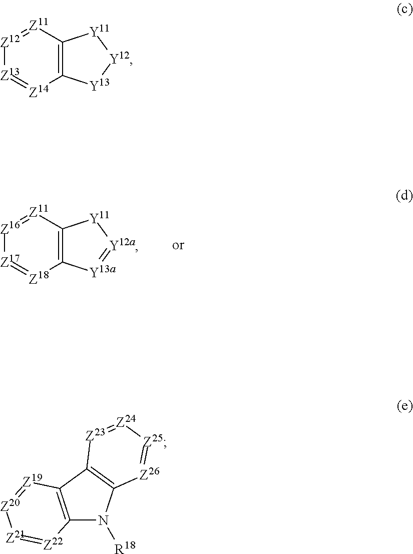

Z¹¹, Z¹², Z¹³, and Z¹⁴ are independently selected from the group consisting of C and —C(R^{3b}); provided that zero or one of Z¹¹, Z¹², Z¹³, and Z¹⁴ is C;

Z¹⁵, Z¹⁶, Z¹⁷, and Z¹⁸ are independently selected from the group consisting of C and —C(R^{3b}); provided that zero or one of Z¹⁵, Z¹⁶, Z¹⁷, and Z¹⁸ is C;

Z¹⁹, Z²⁰, Z²¹, Z²², Z²³, Z²⁴, Z²⁵, and Z²⁶ are independently selected from the group consisting of C and —C(R^{3c}); provided that one of Z¹⁹, Z²⁰, Z²¹, Z²², Z²³, Z²⁴, Z²⁵, and Z²⁶ is C and the group of formula (e) is attached to Ar¹¹ through the C atom;

Y¹¹ at each occurrence is independently selected from the group consisting of O, S, —N(R₁₂), —C(R¹³), and —C(R¹³)(R^{13a});

Y¹² is selected from the group consisting of —N(R¹²), C(═O), —C(R¹³), and —C(R¹³)(R^{13a});

Y¹³ is selected from the group consisting of —N(R¹²), —C(R¹³), and —C(R¹³)(R^{13a}); provided that zero or one of Y¹¹, Y¹², and Y¹³ is —C(R¹³) in a group of formula (c);

wherein when one of Y¹¹, Y¹², and Y¹³ is —C(R¹³) in a group of formula (c), then Z¹¹, Z¹², Z¹³, and Z¹⁴ are each —C(R^{13b}) and the group of formula (c) is attached to Ar¹¹ through the C atom of —C(R¹³) of Y¹¹, Y¹², or Y¹³; and also when one of Z¹¹, Z¹², Z¹³, and Z¹⁴ is C, then Y¹¹, Y¹², and Y¹³ are other than —C(R¹³) and the group of formula (c) is attached to Ar¹¹ through the C atom of Z¹¹, Z¹², Z¹³, or Z¹⁴;

Y^{12a} and Y^{13a} are independently selected from the group consisting of N, C and —C(R^{13a}); provided that when Y¹¹ is —C(R¹³) in a group of formula (d), Y^{12a} and Y^{3a} are selected from the group consisting of N and —C(R¹³), and when one of Y^{12a} and Y^{13a} is C, then Y¹¹ in a group of formula (d) is O, S, —N(R¹²), or —C(R¹³)(R^{13a});

wherein when one of Z¹⁵, Z¹⁶, Z¹⁷, and Z¹⁸ is C, then Y¹¹ in a group of formula (d) is selected from the group consisting of O, S, —N(R₁₂), and —C(R¹³)(R^{13a}); Y^{12a} and Y^{13a} are each independently selected from the group consisting of N and —C(R^{13a}); and the group of formula (b) is attached to Ar¹¹ through the C of Z¹⁵, Z¹⁶, Z¹⁷ or Z¹⁸; and also wherein when Y¹¹ in a group of formula (d) is —C(R¹³) or one of Y^{12a} and Y^{13a} is C, then Z¹⁵, Z¹⁶, Z¹⁷, and Z¹⁸ are each —C(R^{13b}) and the group of formula (d) is attached to Ar¹¹ through the C atom of —C(R¹³) of Y¹¹ in the group of formula (d) or through the C atom of Y^{12a} or Y^{13a};

R¹¹ and R¹² at each occurrence are each independently selected from the group consisting of hydrogen and alkyl;

R¹³ and R^{13a} at each occurrence are each independently selected from the group consisting of hydrogen, halogen, alkyl, aryl, —OR, —NR¹⁵R¹⁶, -alkyl-OR¹⁴, and -alkyl-NR¹⁵R¹⁶;

R^{13b} and R^{13c} at each occurrence are each independently selected from the group consisting of hydrogen, halogen, alkyl, aryl, —OR¹⁴, —NR¹⁵R¹⁶, -alkyl-OR¹⁴, -alkyl-NR¹⁵R¹⁶, and —SCN;

R¹⁴ is selected from the group consisting of hydrogen, alkyl, aryl, alkylcarbonyl, and arylcarbonyl;

R¹⁵ and R¹⁶ at each occurrence are each independently selected from the group consisting of hydrogen, alkyl, aryl, alkylcarbonyl, alkoxycarbonyl, aryloxycarbonyl, and arylcarbonyl, provided that at least one of R¹⁵ and R¹⁶ is hydrogen or alkyl; and

R¹⁸ is selected from the group consisting of hydrogen and alkyl.

One manner of preparing fused bicycloheterocycle substituted quinuclidine derivatives involves treating 3-quinuclidinol with a bromo-, chloro-, or iodo-substituted halophenyl iodide, Cul, Cs₂CO₃, and 1,10-phenanthroline as described in Org. Lett., 2002, 4, 973, to obtain a halophenoxy quinuclidine derivative. The resulting halophenoxy quinuclidine derivative can be treated with bis(pinacolato)diboron or bis(catecholato)diboron in the presence of a palladium catalyst to provide the corresponding tin or boronic acid, which is reacted with a halide of a fused bicycloheterocycle to afford a fused bicycloheterocycle substituted ether. Fused bicycloheterocycle substituted amines and fused bicycloheterocycle substituted thioethers can be prepared in a similar manner, but substituting known starting materials for the 3-quinuclidinol and halophenyl iodide, for example reacting 3-quinuclidinone and with a halo-substituted aniline to obtain fused bicycloheterocycle substituted amines or reacting 3-chloroquinuclidine with a halobiarylthiol to obtain fused bicycloheterocycle substituted thioethers. Further description for preparing fused bicycloheterocycle substituted quinuclidine derivatives can be found in US Publication Nos. US20050137184, published on Jun. 23, 2005; US20050137204, published on Jun. 23, 2005; and US20050245531, published on Nov. 3, 2005, each of which is hereby incorporated by reference in its entirety.

Spirocyclic quinuclidinic ether derivatives; 3-quinuclidinyl amino-substituted biaryl derivatives, 3-quinuclidinyl heteroatom-bridged biaryl derivatives; and amino-substituted tricyclic derivatives also can be prepared and are suitable for the present invention. Further description for preparing such compounds can be found in International Publication Nos. WO 2005/066168, published on Jul. 21, 2005; WO 2005/066166, published on Jul. 21, 2005; WO 2005/066167, published on Jul. 21, 2005; and WO 2005/077899, published on Aug. 25, 2005, each of which is hereby incorporated by reference in its entirety.

Examples of compounds reported as α7 agonists or partial agonists are quinuclidine derivatives, for example as described in WO 2004/016608 and WO 2004/022556; and tilorone derivatives, for example also as described in WO 2004/016608.

Examples of compounds reported as positive allosteric modulators are 5-hydroxyindole analogs, for example as described in WO 01/32619, WO 01/32620, WO 01/32622; tetrahydroquinoline derivatives, for examples as described in WO 04/098600; amino-thiazole derivatives; and diarylurea derivatives, for example as described in WO 04/085433.

Specific examples of compounds that are suitable neuronal nicotinic subtype α7 receptor ligands include, but are not limited to:
5-(6-[(3R)-1-azabicyclo[2.2.2]oct-3-yloxy]pyridazin-3-yl)-1H-indole;
2-(6-phenylpyridazine-3-yl)octahydropyrrolo[3,4-c]pyrrole;
5-[5-{(1R,5R)-6-methyl-3,6-diaza-bicyclo[3.2.0]hept-3-yl}-pyridin-2-yl]-1H-indole; and
5-[6-(cis-5-methyl-hexahydro-pyrrolo[3,4-c]pyrrol-2-yl)-pyridazin-3-yl-1H-indole.

Compounds modulating activity of nicotinic acetylcholine receptor α7 subtype are suitable for the invention regardless of the manner in which they affect the receptor. Other compounds reported as demonstrating α7 activity include, but are not limited to, quinuclidine amide derivatives, for example PNU-282987, N-[(3R)-1-azabicyclo[2.2.2]oct-3-yl]-4-chlorobenzamide, and others as described in WO 04/052894, and MEM-3454. Additional compounds can include, but are not limited to, AR R17779, AZD0328, WB-56203, SSR-180711A, GTS21, and OH-GTS-21, which are all described in the publicly available literature. Yet other compounds that are reportedly under investigation that demonstrate α7 are TC-5619 and varenicline. Further information on TC-5619 can be obtained from Targacept. Further information on varenicline can be obtained from Pfizer.

In addition to the specific compounds, one of ordinary skill in the art would readily recognize that a variety of pharmaceutically acceptable salts, esters, and amides of a parent compound also can be incorporated into a composition, method, or article of manufacture of the present invention.

Suitable pharmaceutically acceptable basic addition salts include, but are not limited to cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Other possible compounds include pharmaceutically acceptable amides and esters. “Pharmaceutically acceptable ester” refers to those esters which retain, upon hydrolysis of the ester bond, the biological effectiveness and properties of the carboxylic acid and are not biologically or otherwise undesirable. For a description of pharmaceutically acceptable esters as prodrugs, see Bundgaard, E., ed., (1985) Design of Prodrugs, Elsevier Science Publishers, Amsterdam, which is hereby incorporated by reference. These esters are typically formed from the corresponding carboxylic acid and an alcohol. Generally, ester formation can be accomplished via conventional synthetic techniques. (See, e.g., March Advanced Organic Chemistry, 3rd Ed., John Wiley & Sons, New York p. 1157 (1985) and references cited therein, and Mark et al. Encyclopedia of Chemical Technology, John Wiley & Sons, New York (1980), both of which are hereby incorporated by reference. The alcohol component of the ester will generally comprise (i) a C2-C12 aliphatic alcohol that can or can not contain one or more double bonds and can or can not contain branched carbons or (ii) a C7-C12 aromatic or heteroaromatic alcohols. This invention also contemplates the use of those compositions which are both esters as described herein and at the same time are the pharmaceutically acceptable salts thereof.

“Pharmaceutically acceptable amide” refers to those amides which retain, upon hydrolysis of the amide bond, the biological effectiveness and properties of the carboxylic acid and are not biologically or otherwise undesirable. For a description of pharmaceutically acceptable amides as prodrugs, see Bundgaard, H., Ed., (1985) Design of Prodrugs, Elsevier Science Publishers, Amsterdam. These amides are typically formed from the corresponding carboxylic acid and an amine. Generally, amide formation can be accomplished via conventional synthetic techniques. (See, e.g., March Advanced Organic Chemistry, 3rd Ed., John Wiley & Sons, New York, p. 1152 (1985) and Mark et al. Encyclopedia of Chemical Technology, John Wiley & Sons, New York (1980), both of which are hereby incorporated by reference. This invention also contemplates the use of those compositions which are amides, as described herein, and at the same time are the pharmaceutically acceptable salts thereof.

It also will be readily apparent to one with skill in the art that the compounds can be generated in vivo by administration of a drug precursor which, following administration, releases the drug in vivo via a chemical or physiological process (e.g., a parent compound on being brought to the physiological pH or through enzyme action is converted to the desired drug form).

### Administration

As noted above, it has been discovered that psychotic conditions can be treated by concurrently administering to a patient (i.e. a human) in need thereof, an antipsychotic and an α7 receptor ligand. It has been discovered that such combination is especially useful in expanding the dosage range and reducing the incidence of EPS.

As used in this application, the term “concurrent administration” refers to administering the α7 receptor ligand to a patient, who has been prescribed (or has consumed) at least one antipsychotic, at an appropriate time so that the patient's symptoms may subside. This may mean simultaneous administration of the α7 receptor ligand and the antipsychotic, or administration of the medications at different, but appropriate times. Establishing such a proper dosing schedule will be readily apparent to one skilled in the art, such as a psychiatrist, or other physician.

The dosage range at which the antipsychotic and the α7 receptor ligand will be administered concurrently can vary widely. The specific dosage will be chosen by the patient's physician taking into account the particular antipsychotic chosen, the seventy of the patient's illness, any other medical conditions or diseases the patient is suffering from, other drugs the patient is taking and their potential to cause an interaction or adverse event, the patient's previous response to antipsychotic medication, and other factors.

The antipsychotic and the α7 receptor ligand should be administered concurrently in amounts that are effective to treat the patient's schizophrenia or related condition. In more general terms, one would create a combination of the present invention by choosing a dosage of an antipsychotic and a dosage of the α7 receptor ligand according to the spirit of the guidelines presented above.

The antipsychotic therapy of the present invention is carried out by administering an antipsychotic together with an α7 receptor ligand in any manner which provides effective levels of the compounds in the body at the same time. Typically, the combination will be administered orally.

However, the invention is not limited to oral administration. The invention should be construed to cover any route of administration that is appropriate for the medications involved and for the patient. For example, transdermal administration may be very desirable for patients who are forgetful or petulant about taking oral medicine. Injections may be appropriate for patients refusing their medication. One of the drugs may be administered by one route, such as oral, and the others may be administered by the transdermal, percutaneous, intravenous, intramuscular, intranasal, or intrarectal route, in particular circumstances. The route of administration may be varied in any way, limited by the physical properties of the drugs and the convenience of the patient and the caregiver.

The following examples are being presented to further illustrate the invention. They should not be construed as limiting the invention in any manner. The dosage range of the currently available antipsychotics can be broad. Treatment-limiting side effects such as EPS are dose related, as previously described. Therefore, as an example, typical dose ranges for some commonly used antipsychotics are below. This list is not intended to be complete but is merely an illustration of current clinical usage and its correlation with EPS risk.

TABLE 1
Currently used Antipsychotic Drugs,
Dose Ranges, and Side Effect Profiles

| Antipsy- | Clinical Dose Range | | |
|---|---|---|---|
| chotic | (common/recom- | | Other Dose |
| medica- | mended dose | | Related |
| tion | (full reported range)) | EPS risk | Side Effects |
| Haloperidol | 2-5 mg/kg per day | ++++ | hyperprolac- |
| | (0.5 to 10 mg/kg per | | tinaemia; |
| | day) | | sexual |
| | | | dysfunction |
| Risperidone | 2-8 mg/kg per day | ++ | hyperprolac- |
| | (0.25 to 16 mg/kg per | (augmented | tinaemia; |
| | day) | risk >6 | sexual |
| | | mg/kg/day) | dysfunction |
| Olanzapine | 1-30 mg/kg per day | + | |
| | (0.25 to 100 mg/kg per | (augmented | |
| | day) | risk >7.5 | |
| | | mg/kg/day) | |
| Clozapine | 200-600 mg/kg per day | +/− | seizures (risk of |
| | (12.5 to 900 mg/kg per | | seizures aug- |
| | day) | | mented >600 |
| | | | mg/kg/day); |
| Quetiapine | >500 mg/kg per day | +/− | somnolence; |
| | (150 to 750 mg/kg per | | increased |
| | day) | | triodothyronine |
| | | | an thyroxine |
| | | | levels |
| Ziprasidone | 80-160 mg/kg per day | ++ | |
| | (4-160 mg/kg per | (augmented | |
| | day) | risk >80 | |
| | | mg/kg/day) | |
| Arpiprazole | 10-15 mg/kg day | +/− | somnolence |
| | (10-20 mg/kg per | | |
| | day) | | |

Table 1 references include Practice Guidelines for the Treatment of Psychiatric Disorders Compendium 2002, American Psychiatric Assoc., Washington, D.C., 2002; Kapur and Remington, Ann. Rev. Med, 2001, 52:503-517; Kinon et al, CNS Drugs, 2004, 18:597-616; Tarsy et al, CNS Drugs, 2002, 16:23-45; Kulisevsky and Otermin, Neurologia, 2003, 18:262-268; Davis and Chen; J Clin Psychopharmacol, 2004, 24, 192-208)

An agent that meaningfully enhances efficacy of an antipsychotic agent without alone causing adverse effects (e.g. extrapyramidal effects) or exaggerating the side effect profile of the antipsychotic agent, such as an α7 receptor ligand, should enhance the therapeutic window of the antipsychotic agent. There is no known prior knowledge for the use of α7 neuronal nicotinic receptor ligands (agonists, antagonists or allosteric modulators) as adjunctive therapy to increase the therapeutic window by enhancing alleviation of positive symptoms without exacerbating side effects. However, the ability of an adjunctive agent, i.e. α7 agonist or modulator to increase the potency and efficacy of an antipsychotic would potentially enhance the clinical utility of the antipsychotic by increasing the therapeutic window in which the clinician can titrate the dose. This would be relevant both for the typical antipsychotics that have the greatest EPS liability where the increased therapeutic window may be small but meaningful as well as for atypical antipsychotics that show EPS at higher doses where the increased therapeutic window could be expected to be substantially larger.

Accordingly, in the present invention, an antipsychotic is used in combination with an α7 receptor ligand, and can be administered at a lower dose, including a sub efficacious dose to have a better effect, and to eliminate or reduce the incidence of antipsychotic related side effects commonly encountered in the clinic.

TABLE 2
Example Dose Range Determinations for Antipsychotics

| | | | | | | Decrease in |
|---|---|---|---|---|---|---|
| | Maximum | | | | | Common |
| | Dose in | | Optimal Dose | | Dose of | Dose to |
| | Common | | and/or | | Increased | Meaningfully |
| Antipsychotic | Clinical | | Minimally | | Side Effect | Impact Side |
| Medication | Range | | Effective Dose | | Risk | Effects |
| Haloperidol | 5 | mg/day | 2-5 | mg/day | ≈3 mg (>78% | 50% |
| | | | | | D₂ occupancy | |
| | | | | | increases risk | |
| | | | | | for EPS) | |
| Risperidone | 8 | mg/day | 4-6 | mg/day | ≧6 mg/day | 25-50% |
| | | | | | (EPS risk) | |
| Olanzapine | 30 | mg/day | 10-20 | mg/day | | 33% |
| Clozapine | 600 | mg/day | 300-400 | mg/day | <300 mg/day | 33-50% |
| | | | | | (low risk for | |
| | | | | | seizures) | |
| | | | | | 300-599 mg/day | |
| | | | | | (moderate risk | |
| | | | | | for seizures) | |
| Ziprasidone | 160 | mg/day | 120 | mg/day | >80 mg/day | 25-50% |
| | | | | | (EPS risk) | |
| Arpiprazole | 20 | mg/day | 15 | mg/day | dose | 25% |
| | | | | | dependent | |
| | | | | | somnolence | |

References for Table 2: Practice Guidelines for the Treatment of Psychiatric Disorders Compendium 2002, American Psychiatric Assoc., Washington D.C., 2002; Kapur, et al., Am. J. Psychiatry (2000) vol. 157:514-20.

To meaningfully reduce risk of dose dependent side effects associated with antipsychotics, when α7 receptor ligands are added to the therapy, the doses of the antipsychotics would be reduced by 25-50% and/or limited to 25-50% of standard maximum doses used in common practice. At these doses, the patient would retain full antipsychotic efficacy against positive symptoms but at lower risk for side effects such as EPS.

The term “effective amount” as used herein refers to a sufficient amount of the individual compound to treat or prevent anxiety disorders, mood disorders, and psychotic disorders or the condition to be treated at a reasonable benefit/risk ratio in the judgment of the administering specialist applicable to any medical treatment.

The term “sub efficacious” as used herein, for example to refer to a “sub efficacious dose” or a “sub efficacious amount” refers to a dose or amount of the individual compound less than an amount for treating or preventing anxiety disorders, mood disorders, psychotic disorders or the condition to be treated at a reasonable benefit/risk ratio in the judgment of the administering specialist applicable to the medical treatment.

The term “maximally efficacious” as used herein, for example to refer to a “maximally efficacious dose” or a “maximally efficacious amount” refers to a dose or amount of the individual compound having the greatest effect for treating or preventing anxiety disorders, mood disorders, psychotic disorders or the condition to be treated at a reasonable benefit/risk ratio in the judgment of the administering specialist applicable to the medical treatment.

The specific effective dose level for any particular patient will depend upon a variety of factors including the disorder being treated and the severity of the disorder; activity of the specific compound employed; the specific composition employed; the age. However, some variation in dosage will necessarily occur depending upon the condition of the subject being treated. The person responsible for administration will, in any event, determine the appropriate dose for the individual subject.

The exact formulation, route of administration, and dosage can be chosen by the individual physician in view of the patient's condition. Dosage amount and interval can be adjusted individually to provide plasma levels of the active moiety which are sufficient to maintain therapeutic effects.

The following dosage amounts and other dosage amounts set forth elsewhere in this description and in the appendant claims are for an average human subject having a weight of about 65 kg to about 70 kg. The skilled practitioner will readily be able to determine the dosage amount required for a subject whose weight falls outside the 65 kg to 70 kg range, based upon the medical history of the subject. All doses set forth herein, and throughout the appendant claims, if applicable, are daily doses.

The suitable amount of antipsychotic drug is based on recommended dose range, preferably at the low end, for example as illustrated in Table 2, and combined with an effective dose of the α7 receptor ligand. The effective dose range of the α7 receptor ligand will be adjusted to ensure efficacious plasma levels judged from clinical trials and can range depending on the duration of administration (once or twice daily or sustained release) of the product, as recommended by the manufacturer.

### Formulations

The antipsychotic and α7 receptor ligand compounds can be administered as a single pharmaceutical composition, or separately to achieve a concomitant or controlled effect. Such compositions may take any physical form that is suitable for pharmaceuticals. Pharmaceutical compositions suitable for oral administration are particularly preferred. Such pharmaceutical compositions contain an effective amount of each of the compounds, which effective amount is related to the daily dose of the compounds to be administered. Each dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the antipsychotic therapy is being given.

The composition contains at least one pharmaceutically acceptable excipient, or inert ingredient. The inert ingredients and manner of formulating the pharmaceutical compositions are conventional, except for the presence of the combination of the present invention. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The amount of the compounds, however, is best defined as the effective amount, that is, the amount of each compound which provides the desired dose to the patient in need of such treatment. The specific combination of any antipsychotic and α7 receptor ligand compound or compounds can be chosen and formulated solely for convenience and economy. Any of the combinations may be formulated in any desired form of composition. Some examples of compositions are described herein, followed by some typical formulations.

Capsules are prepared by mixing the compounds with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours, and similar edible powders.

If desired, the capsules can be formulated so that the contents are removed from the capsules prior to ingestion by the patient. The capsule contents may be diluted in foods, juices, or other substance, in order to simplify administration to those who have difficulty swallowing. Methods for manufacturing such a dosage form would be readily apparent to one skilled in the art.

The medications may also be formulated into liquids or syrups, as is known in the art, in order to simplify administration. The medication can be dissolved in or added to liquids, flavorants, antioxidants, stabilizers, or other inactive ingredients, as is known in the art. Such dosage forms have particular suitability with the elderly, such as dementia patients.

Tablets are prepared by direct compression, by wet granulation, or by dry granulation. Their formulations usually incorporate diluents, binders, lubricants, and disintegrators as well as the compound. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant is necessary in a tablet formulation to prevent the tablet and punches from sticking in the die. The lubricant is chosen from such slippery solids as talc, magnesium and calcium stearate, stearic acid, and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp, and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer which is insoluble in acid environments, and soluble in basic environments. Exemplary films are cellulose acetate phthalate, polyvinyl acetate phthalate, hydroxypropyl methylcellulose phthalate, and hydroxypropyl methylcellulose acetate succinate.

Tablets are often coated with sugar as a flavor and sealant. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches also are suitable for administering the combination. Typically transdermal patches comprise a resinous composition in which the drugs will dissolve, or partially dissolve, which is held in contact with the skin by a film which protects the composition. More complicated patch compositions are also in use, particularly those having a membrane pierced with innumerable pores through which the drugs are pumped by osmotic action.

### Packaging

To enhance patient convenience, any antipsychotic and α7 receptor ligand may be formulated into a single dosage form. Alternatively, separate dosage forms can be used, yet packaged in a single container for dispensing by the pharmacist, for example, as with a blister pack. Such packaging is typically designed to help a patient comply with a dosage regimen and to consume all of the required medication.

An article of manufacture, typically refers to the packaging, can comprise a first pharmaceutical dosage form with an antipsychotic and a second pharmaceutical dosage form with an α7 receptor ligand. The article of manufacture can contain a first and second pharmaceutical dosage form in a single dosage form or as separate dosage forms.

Examples of such packaging are well known to those skilled in the pharmaceutical arts. For example, Pfizer distributes an antibiotic known as Zithromax®. Patients must consume 2 pills on the first day and one pill after that for 4 days in order to eradicate the infection. To allow a patient to comply with such a complicated schedule, Pfizer packages the medication in a blister pack that is commonly referred to as a Z-pack. Similar packages are used with steroids in which the dosage must be tapered. Birth control pills are another example of packaging pharmaceuticals to enhance convenience.

The antipsychotic and α7 receptor ligand may be incorporated into such packaging to enhance patient convenience. If desired, such packaging may be used even if the antipsychotic and α7 receptor ligand are in a single dosage form. The particulars of such packaging will be readily apparent to one skilled in the art.

As is well-known to those skilled in the art, the packaged pharmaceutical will include an insert. Such insert describes the drugs, their doses, possible side effects and indication. Thus, the present invention should be construed to include a package containing at least one antipsychotic compound in combination with at least one α7 receptor ligand. The compounds may be in a single or separate dosage forms.

### Psychotic Disorders

As noted above, the combination of an antipsychotic and an α7 receptor ligand will have efficacy in psychoses and other disorders or mental illnesses besides schizophrenia.

For example, schizophreniform is a condition exhibiting the same symptoms as schizophrenia, but is characterized by an acute onset with resolution in two weeks to six months. Often, schizophreniform is used to describe a patient's first schizophrenic episode. The patient presents with symptoms identical to those seen in the acute phase of schizophrenia, but the patient has no previous history of schizophrenia. Clinicians also refer to schizophreniform as “early schizophrenia”. Treatment for schizophreniform disorder can be accomplished in the manner as previously described for the administration and formulation of the invention.

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; brief psychotic disorder; shared psychotic disorder; psychotic disorder due to a general medical condition; substance-induced psychotic disorder, for example psychosis induced by alcohol, amphetamine, cannabis, cocaine, hallucinogens, inhalants, opioids, or phencyclidine; personality disorder of the paranoid type; personality disorder of the schizoid type; psychotic disorder not otherwise specified.

The meanings attributed to the different types and subtypes of psychotic disorders are as stated in DSM-IV-TR. (Diagnostic and Statistical Manual of Mental Disorders, 4th ed., American Psychiatric Assoc., Washington, D.C., 2002, p. 297-343).

Schizophrenia as used herein refers to a disorder that lasts for at least 6 months and includes at least one month of active-phase symptoms (i.e., two [or more] of the following: delusions, hallucinations, disorganized speech, grossly disorganized or catatonic behavior, negative symptoms) (Diagnostic and Statistical Manual of Mental Disorders, DSM-IV-TR, 4th ed., American Psychiatric Assoc., Washington, D.C., 2002).

Schizoaffective disorder is defined as a disorder in which a mood episode and the active-phase symptoms of schizophrenia occur together and were preceded or are followed by at least 2 weeks of delusions or hallucinations without prominent mood symptoms (Diagnostic and Statistical Manual of Mental Disorders, DSM-IV-TR, 4th ed., American Psychiatric Assoc., Washington, D.C., 2002).

Schizophreniform disorder is defined as a disorder characterized by a symptomatic presentation that is equivalent to schizophrenia except for its duration (i.e., the disturbance lasts from 1 to 6 months) and the absence of a requirement that there be a decline in functioning (Diagnostic and Statistical Manual of Mental Disorders, DSM-IV-TR, 4th ed., American Psychiatric Assoc., Washington, D.C., 2002).

Schizotypical disorder is defined as a lifetime pattern of social and interpersonal deficits characterized by an inability to form close interpersonal relationships, eccentric behavior, and mild perceptual distortions.

The present invention can be used to treat other psychotic disorders such as delusional disorder; brief psychotic disorder; shared psychotic disorder; substance-induced psychotic disorder, for example psychosis induced by alcohol, amphetamine, cannabis, cocaine, hallucinogens, inhalants, opioids, or phencyclidine; psychotic disorder due to a general medical condition; personality disorder of the paranoid type; personality disorder of the schizoid type; and psychotic disorder not otherwise specified.

For example, treating schizophrenia, schizophreniform, or schizoaffective disorder, as used herein also encompasses treating one or more symptoms (positive, negative, and other associated features) of said disorders, for example treating, delusions, or hallucinations, or any such symptoms associated therewith. Other examples of symptoms of schizophrenia and schizophreniform and schizoaffective disorders include disorganized speech, affective flattening, alogia, anhedonia, inappropriate affect, dysphoric mood (in the form of, for example, depression, anxiety or anger), and some indications of cognitive dysfunction.

Delusional disorder as referred to herein is characterized by at least 1 month of nonbizarre delusions without other active-phase symptoms of schizophrenia. (Diagnostic and Statistical Manual of Mental Disorders, DSM-IV-TR, 4th ed., American Psychiatric Assoc., Washington, D.C., 2002).

Brief psychotic disorder is a disorder that lasts more than 1 day and remits by 1 month. (Diagnostic and Statistical Manual of Mental Disorders, DSM-IV-TR, 4th ed., American Psychiatric Assoc., Washington, D.C., 2002).

Shared psychotic disorder is characterized by the presence of a delusion in an individual who is influenced by someone else who has a longer-standing delusion with similar content. (Diagnostic and Statistical Manual of Mental Disorders, DSM-IV TR, 4th ed., American Psychiatric Assoc., Washington, D.C., 2002).

Psychotic disorder due to a general medical condition is characterized by psychotic symptoms judged to be a direct physiological consequence of a general medical condition. (Diagnostic and Statistical Manual of Mental Disorders, DSM-IV-TR, 4th ed., American Psychiatric Assoc., Washington, D.C., 2002).

Psychotic disorder not otherwise specified is a psychotic presentation that does not meet the criteria for any of the specific psychotic disorders defined in the DSM-IV-TR (American Psychiatric Assoc., Washington, D.C., 2002).

In another embodiment, the compounds used in the present invention are useful to treat other disorders that may present with psychotic symptoms as associated features such as dementia of the Alzheimer's type; substance-induced delirium; and major depressive disorder with psychotic features.

In a preferred embodiment, the compounds used in the present invention are useful for treating schizophrenia, a schizoaffective disorder, schizophreniform disorder, or a schizotypical disorder.

The present invention also may be used to treat mood disorders, formerly designated as “affective disorders.” Although mood disorders are not a clearly delineated group of illnesses they include unipolar and bipolar depression, generalized anxiety disorder, and more specific anxiety disorders such as agoraphobia, panic disorder and social phobia, obsessive-compulsive disorder and post traumatic stress disorder (PTSD). There is a high level of similarity and co-morbidity between these illnesses and clinicians may consider them as a single group.

The meanings attributed to the different types and subtypes of mood disorders are as stated in DSM-IV-TR under depressive disorders (“unipolar depression”) and bipolar disorders, generalized anxiety disorder, and more specific anxiety disorders such as agoraphobia, panic disorder and social phobia, obsessive-compulsive disorder and post traumatic stress disorder (PTSD), the contents of which are incorporated by reference herein. (Diagnostic and Statistical Manual of Mental Disorders”, 4th ed., American Psychiatric Assoc., Washington, D.C., 2002, p. 345-484).

The term “affective disorder” as used herein is interchangeable with the term “mood disorders” and refers to disorders that are characterized by changes in mood as the primary clinical manifestation, for example, depression.

The following Examples are provided to illustrate various aspects of the invention and should not be construed as limiting the invention in any manner.

### EXAMPLES

### Example 1

### Clinically Used Typical and Atypical Antipsychotics Agents are Effective in the DBA Mouse Model

Dysfunctions of sensory gating and information processing have been putatively associated with clinical features such as perpetual aberrations, hallucinations and distraction and been considered as potential precursors of sensory overload, cognitive fragmentation and disorganization. Among the physiological measures, one approach has involved the startle reflex with the prepulse inhibition (PPI) paradigm. Patients with schizophrenia exhibit deficits in prepulse inhibition (PPI) of startle, which is linked to both positive and negative symptoms. PPI refers to a reduction in the acoustic startle reflex to a loud noise when the loud noise is preceded by a weak auditory stimulus. At short lead intervals, this has the effect of markedly reducing or gating the amplitude of the startle response and increasing its latency. Disruption of PPI occurs with agonists of dopamine and serotonin, and with glutamate/N-methyl D aspartate (NMDA) receptor antagonists, or can be induced by genetic (e.g., DBA2 mouse strain) and experimental manipulations (e.g., rearing rats in isolation, neurotoxic lesions, or other methods). The naturally occurring deficit in PPI in the DBA2 mouse strain has been shown to be an effective model to evaluate antipsychotic agents and increases in baseline PPI has been observed with clinically effective antipsychotics such as haloperidol, risperidone, and clozapine. (Olivier B., et al., Psychopharmacology (2001) vol. 156:284-290; Ouagazzal A-M., Psychopharmacology (2001) vol. 156:273-283; Simosky J. K., Psychopharmacology (2003) vol. 165:386-396). To accomplish the study, the following materials and methods were used.

Animals:

Male DBA/2J mice (Jackson Laboratories AX9 facility, Bar Harbor, Me., USA) at age 6-8 weeks old were used for all investigations. They were housed under standard facility conditions in groups of eight on a 12 h light/dark cycle (lights on at 0600 h) with ad libitum access to food and water.

Chemicals:

Haloperidol, (4-(4-[4-chlorophenyl]-4-hydroxy-1-piperidinyl)-1-(4-fluorophenyl)-1-butanone, having a molecular weight (MW) of 375.9) was obtained from Sigma-Aldrich (St. Louis, Mo., USA); clozapine (8-chloro-11-(4-methyl-1-piperazinyl)-5H-dibenzo[b,e][1,4]diazepine, MW 326.83) was obtained from Tocris (Ellisville, Mo., USA); risperidone (3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one, MW 410.5) was obtained from ICN Biomedicals Inc. (Aurora, Ohio, USA).

Preparation of Compounds:

Haloperidol, clozapine and risperidone were all solubilized in water/acetic acid, and pH normalized to 5.5 with NaOH. All compounds were administered in solution in a volume of 0.1 ml/10 g body weight.

Experimental Procedure:

Startle response and PPI were measured using startle chambers from Hamilton Kinder (Poway, Calif., USA). Each chamber contained a plexiglas rectangle with an adjustable ceiling housed in a ventilated, sound-attenuated cubicle. The ceiling was adjusted on an individual (animal by animal) basis to allow for adequate headroom but no rears or extensive locomotion. The chamber was placed over an anchor plate attached to a piezoelectric disk to transduce startle responses to a computer. A loudspeaker located in each chamber delivered the background noise (65 dB), and the acoustic stimuli. A constant white noise was maintained in the experimental room for the duration of the experiment by a white noise generator (Radioshack, USA). Each session was initiated with a 5-minute acclimation period followed by four successive 120 dB, 40 ms trials. These trials were not included in the main analysis, but are referred to as baseline responses. Animals were then presented with 5 different trial types: startle pulse (120 dB, 40), or prepulse stimulus of one of three sound levels (70, 75, or 80 dB) for 20 ms, followed 100 ms later by an acoustic startle (120 dB) for 40 ms. A total of 12 trials under each condition were delivered in a random sequence and all trials were separated by a variable inter-trial interval of 5-25 s. Finally, this sequence ended with the presentation of four 120 dB, 40 ms sound bursts (not included in the main analyses, but included in the baseline or habituation analyses). The animals were injected with the test compounds 30 minutes before the start of the trials. In the startle alone trials, the basic auditory startle, or startle response was measured, and in the prepulse plus startle trials, the levels of PPI was calculated as a percentage score for each acoustic prepulse trial type using (typically) the formula: [(startle response for prepulse+pulse)/(startle response for pulse-alone)]*100.

Statistics:

Data were first analyzed using a two-way repeated measures analysis of variance (ANOVA) with two independent factors. If there was a significant interaction of both factors, subsequent post hoc one-way ANOVAs were performed using each treatment combination as an independent group. All post hoc significance was determined using Fishers protected least significant difference test. (p<0.05 was regarded as significant).

Results:

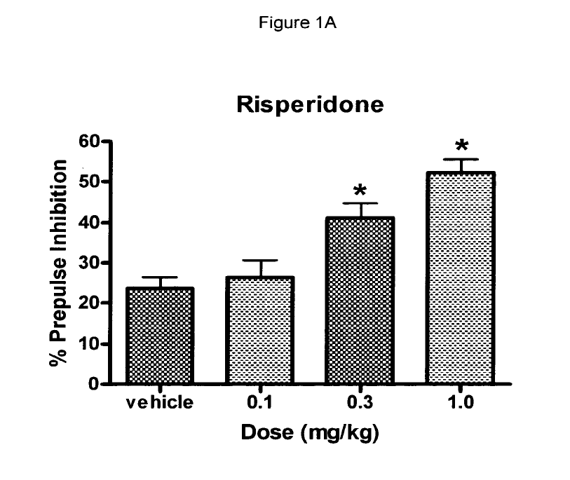

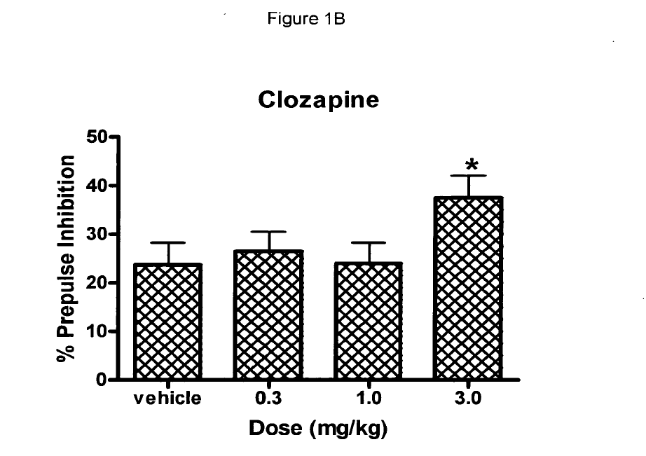

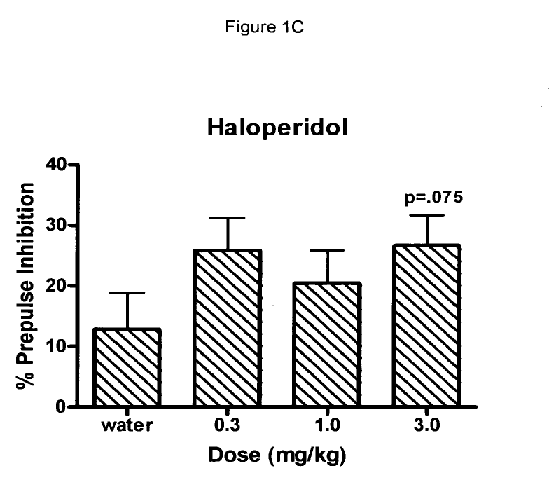

The first series of experiments assessed the ability for antipsychotics, both typical (e.g. haloperidol) and atypical (clozapine and risperidone) antipsychotics in the DBA2 mouse PPI. As shown in FIG. 1, efficacy for haloperidol was observed at 3 mg/kg i.p.; for clozapine at 3 mg/kg i.p., and for risperidone at 0.3 and 1.0 mg/kg i.p. This study indicates that the mouse model (DBA/2 mouse pre-pulse inhibition (PPI) test) is predictive of clinical efficacy against positive symptoms of schizophrenia. (Olivier B, et al., Psychopharmacology (2001) vol 156:284-290.)

### Example 2

### α7 Agonists Potentiated Antipsychotic Effect of Risperidone

To assess the nature of these interactions, the effect of Compound 1 (0.1-10 μol/kg i.p.), an α7 agonist, on a subefficacious dose of risperidone (0.1 mg/kg) was examined. The following materials and methods were used to accomplish the study.

Animals:

Male DBA/2J mice (Jackson Laboratories AX9 facility, Bar Harbor, Me., USA) at age 6-8 weeks old were used for all investigations. They were housed under standard facility conditions in groups of eight on a 12 h light/dark cycle (lights on at 0600 h) with ad libitum access to food and water.

Chemicals:

Compound 1 (lot #1278527), 5-(6-[(3R)-1-azabicyclo[2.2.2]oct-3-yloxy]pyridazin-3-yl-1H-indole, MW 402.32, was prepared at Abbott Laboratories; risperidone (3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one, MW 410.5) was obtained from ICN Biomedicals Inc. (Aurora, Ohio, USA).

Preparation of Compounds:

Compound 1 was solubilized in saline.

Risperidone was solubilized in water+acetic acid, and pH normalized to 5.5 with NaOH. All compounds were administered in solution in a volume of 0.1 ml/10 g body weight.

Experimental Procedure:

Startle response and PPI were measured using startle chambers from Hamilton Kinder (Poway, Calif., USA). Each chamber contained a plexiglas rectangle with an adjustable ceiling housed in a ventilated, sound-attenuated cubicle. The ceiling was adjusted on an individual (animal by animal) basis to allow for adequate headroom but no rears or extensive locomotion. The chamber was placed over an anchor plate attached to a piezoelectric disk to transduce startle responses to a computer. A loudspeaker located in each chamber delivered the background noise (65 dB), and the acoustic stimuli. A constant white noise was maintained in the experimental room for the duration of the experiment by a white noise generator (Radio Shack, USA). Each session was initiated with a 5-minute acclimation period followed by four successive 120 dB, 40 ms trials. These trials are not included in the main analysis, but are referred to as baseline responses. Animals were then presented with 5 different trial types: startle pulse (120 dB, 40), or prepulse stimulus of one of three sound levels (70, 75, or 80 dB) for 20 ms, followed 100 ms later by an acoustic startle (120 dB) for 40 ms. A total of 12 trials under each condition were delivered in a random sequence and all trials were separated by a variable inter-trial interval of 5-25 s. Finally, this sequence ended with the presentation of four 120 dB, 40 ms sound bursts (not included in the main analyses, but included in the baseline or habituation analyses). The animals were injected with the test compounds 30 minutes before the start of the trials. For co-administration studies the α7 agonist was administered 10 minutes before risperidone. Trials were initiated 30 minutes after second injection. In the startle alone trials, the basic auditory startle, or startle response was measured, and in the prepulse plus startle trials, the amount of PPI was calculated as a percentage score for each acoustic prepulse trial type using (typically) the formula: [(startle response for prepulse+pulse)/(startle response for pulse-alone)]*100.

Statistics:

Data were first analyzed using a two-way repeated measures analysis of variance (ANOVA) with two independent factors. If there was a significant interaction of both factors, subsequent post hoc one-way ANOVA was performed using each treatment combination as an independent group. All post hoc significance was determined using Fishers protected least significant difference test. (p<0.05 was regarded as significant).

Results:

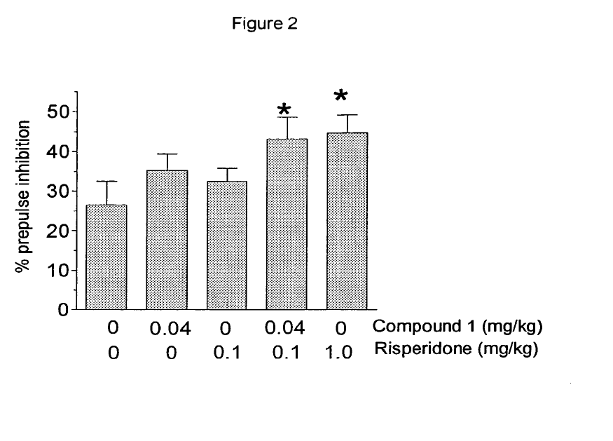

As shown in FIG. 2, Compound 1 alone (at 0.04 mg/kg) does not have an effect, but when combined with a sub efficacious dose of risperidone (0.1 mg/kg), a maximally efficacious response is achieved. Such a response is similar to that achieved by a 10-fold higher dose of risperidone (1.0 mg/kg). The results demonstrate that a dose that is normally weakly efficacious can be made to exhibit robust efficacy upon α7 receptor activation by an agonist. The plasma concentration of Compound 1 required to achieve this effect is less than 100 nM. The study indicates that in a mouse model (DBA/2 mouse pre-pulse inhibition (PPI) test) believed to be predictive of clinical efficacy against positive symptoms of schizophrenia, an α7 receptor ligand increases both the potency and efficacy of an antipsychotic, risperidone. Thus, if such an antispychotic is used in combination with an α7 receptor ligand, then it can be administered at a lower dose, to have a better effect, and to eliminate or reduce the incidence of antipsychotic-related side effects commonly encountered in the clinic. Positive allosteric modulators are compounds that potentiate effects of endogenous (acetylcholine) and exogenous (e.g. Compound 1) agonists on the α7 neuronal nicotinic receptor, and accordingly, such agents would also be expected to have similar effects.

### Example 3

### α7 Agonists Do Not Exhibit Side Effect Profile like Risperidone and Do Not Exacerbate the Cataleptic Effect of Risperidone

One of the adverse effects of antipsychotic medications is extrapyramidal movement disorder syndrome attributed to blockade of the dopamine D₂ receptors. Extrapyramidal movement disorder can be predicted by the cataleptic response elicited by an antipsychotic in a rodent. To assess whether Compound 1 alone evoked cataleptic responses or interfered with the cataleptic effect of the antipsychotic, the following set of studies were conducted. The materials and methods used to accomplish the study follow.

Animals:

Male Sprague Dawley rats (CRL: CD (SD), Charles River Laboratories, Omaha, Nebr.) weighing 300-325 g were used for the experiment. They were housed under standard conditions in groups of 4 rats on a 12 h light/dark cycle (lights on at 0600 h) with ad libitum access to food and water.

Chemicals:

Compound 1 (lot #1278527), MW 402.32 was prepared at Abbott Laboratories; risperidone (3-[2-[4-(6-fluoro-1,2-benzisoxazol-piperidinyl]ethyl]-6,7,8,9-tetrahydro-2-methyl-4H-pyrido[1,2-a]pyrimidin-4-one, MW 410.5) was obtained from ICN Biomedicals Inc. (Aurora, Ohio, USA).

Preparation of Compounds:

Compound 1 was solubilized in saline. Risperidone was solubilized in water+acetic acid, and pH normalized to 5.5 with NaOH. All compounds were administered in solution in a volume of 1.0 ml/kg body weight.

Experimental Procedure:

Rats were handled and habituated to the testing room before starting. On test day rats were transferred into individual cages and left undisturbed for at least one hour. All compounds are dosed at 1.0 ml/kg i.p. In the case of co-treatment, the α7 agonist was administered 10 minutes prior to the risperidone. Rats were tested at 60, 120, 180, and 240 minutes post-injection for cataleptic responses and returned to cages in-between test sessions. The degree of catalepsy was measured by gently placing both forepaws over a metal bar (1.1 cm. diameter suspended 8 cm. above the table top). The time in seconds until the rat took both paws off the bar was recorded, with a maximum cut-off of 300 seconds. The total duration of catalepsy in the different time points was used for analysis. At least 5 trials were attempted on each rat with 5 seconds used as a low-end cut-off for catalepsy (time scored as zero). For catalepsy times between 5-15 seconds, the highest time of 5 trials was recorded. Alternately, any catalepsy trial time that was greater than 15 seconds (up to 300 seconds) was recorded.

Statistics:

Data were first analyzed using a two-way repeated measures analysis of variance (ANOVA) with two independent factors. If there was a significant interaction of both factors, subsequent post hoc one-way ANOVAs were performed using each treatment combination as an independent group. All post hoc significance was determined using a Student's t-test. (p<0.05 was regarded as significant).

Results:

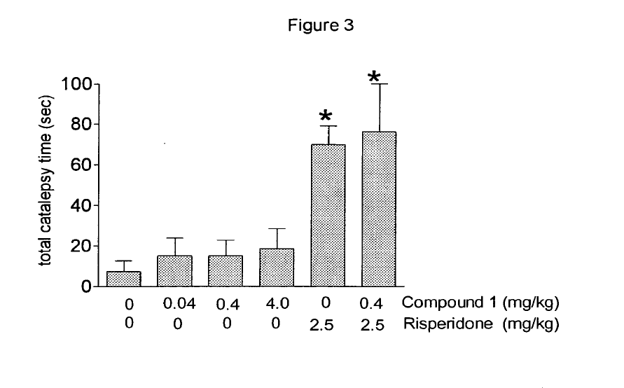

As shown in FIG. 3, Compound 1 at doses ranging from 0.04 mg/kg (a dose that boosted the antipsychotic effects of risperidone shown in Example 2) to 4 mg/kg alone did not induce catalepsy, an EPS predictor. Moreover, Compound 1 also did not alter cataleptic behavior of risperidone (2.5 mg/kg i.p.) when administered in combination. As shown in FIG. 3, risperidone (2.5 mg/kg i.p.) alone evoked cataleptic effects, and addition of Compound 1 did not enhance those effects. This study demonstrates that an α7 agonist does not show EPS effects, and does not have the potential to alter the side effect profile of the antipsychotic drug. However, since efficacy of the antipsychotic drug is improved at comparable doses, the net effect is overall improvement in therapeutic window in combination with α7 neuronal nicotinic receptor ligand.

### Example 4

### α7 Agonist Potentiated Antipsychotic Effect of Haloperidol

The PPI model was used to investigate the effect of a selective α7 nicotinic receptor agonist, Compound 1 (0.04-4.0 mg/kg), on a subefficacious dose of haloperidol.

Animals:

Male DBA/2J mice (Jackson Labs AX9 facility, Bar Harbor, Me., USA) at age 6-8 weeks old were used. They were housed under standard facility conditions in groups of eight on a 12 h light/dark cycle (lights on at 0600 h) with ad libitum access to food and water.

Chemicals:

Compound 1 (lot #1278527), MW 402.32 was prepared at Abbott Laboratories; haloperidol (4-(4-[4-chlorophenyl]-4-hydroxy-1-piperidinyl)-1-(4-fluorophenyl)-1-butanone, MW 375.9) was obtained from Sigma Aldrich (St. Louis, Mo., USA).

Preparation of Compounds:

Compound 1 was solubilized in saline. Haloperidol was solubilized in water+acetic acid, and pH normalized to 5.0 with NaOH. All compounds were administered in solution in a volume of 0.1 ml/10 g body weights.

Experimental Procedure:

Startle response and PPI were measured using startle chambers from Hamilton Kinder (Poway, Calif., USA). Each chamber contained a plexiglas rectangle with an adjustable ceiling housed in a ventilated sound-attenuated cubicle. The ceiling was adjusted on an individual (animal by animal) basis to allow for adequate headroom but no rears or extensive locomotion. The chamber was placed over an anchor plate attached to a piezoelectric disk to transduce startle responses to a computer. A loudspeaker located in each chamber delivered the background noise (65 dB) and the acoustic stimuli. A constant white noise was maintained in the experimental room for the duration of the experiment by a white noise generator (Radio Shack, USA). Each session was initiated with a 5-minute acclimation period followed by four successive 120 dB, 40 ms trials. These trials were not included in the main analysis, but were referred to as baseline responses. Animals were then presented with 5 different trial types. Startle pulse (120 dB, 40 ms), or prepulse stimulus of one of three sound levels (70, 75, or 80 dB) for 20, ms, followed 100 ms later by an acoustic startle (120 dB) for 40 ms, or no stimulus at all. A total of 12 trials under each condition were delivered in a random sequence and all trials were separated by a variable inter-trial interval of 5-25 s. Finally, this sequence ended with the presentation of four 120 dB, 40 ms sound bursts (not included in the main analyses, but included in the baseline or habituation analyses). The animals were injected with the test compounds 30 minutes before the start of the trials. For co-administration studies, the α7 agonist was administered 10 minutes before haloperidol. Trials were initiated 30 minutes after the second injection. In the startle alone trials, the amount of PPI was calculated as a percentage score for each acoustic prepulse trial type using (typically) the formula:

[(startle response for prepulse+pulse)/(startle response for pulse alone)]*100.

Statistics:

Data were first analyzed using a two-way repeated measures analysis of variance (ANOVA) with two independent factors. If there was a significant interaction of both factors, subsequent post hoc one-way ANOVA were performed using each treatment combination as an independent group. All post hoc significance was determined using Dunnett's multiple comparison test (p<0.05 was regarded as significance).

Results:

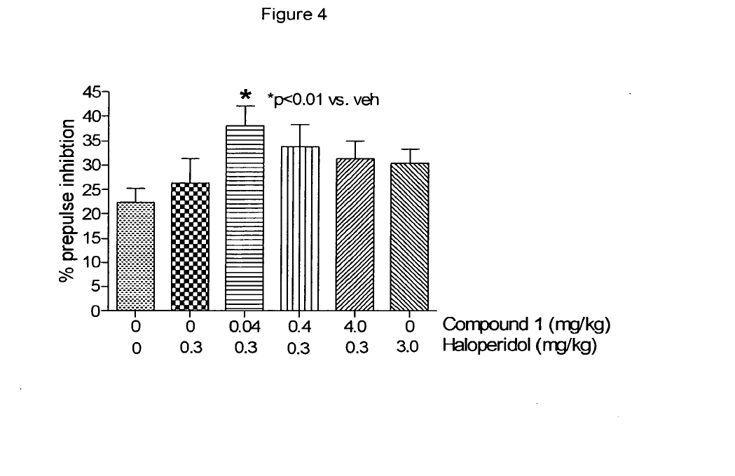

As shown in FIG. 4, haloperidol (0.3 mg/kg) was found to be more efficacious in the presence of Compound 1. The combination of Compound 1 and haloperidol was more efficacious than a higher dose of haloperidol (3 mg/kg) alone. This demonstrates that a dose that is normally weakly efficacious can be made to exhibit robust efficacy upon α7 receptor activation by an agonist. The plasma concentration of Compound 1 required to achieve this effect is about 3 ng/mL (˜10 nM).

### Example 5

### α7 Agonists Do Not Interfere with Efficacy of Haloperidol

To assess whether Compound 1 could attenuate the effect of haloperidol, a study was conducted where Compound 1 (0.04-4.0 mg/kg i.p.) was administered 10 minutes prior to an maximally efficacious dose of haloperidol.

Animals:

Male DBA/2J mice (Jackson Labs AX9 facility, Bar Harbor, Me., USA) at age 6-8 weeks old were used. They were housed under standard facility conditions in groups of eight on a 12 h light/dark cycle (lights on at 0600 h) with ad libitum access to food and water.

Chemicals:

Compound 1 (lot #1278527), MW 402.32 was prepared at Abbott Laboratories; haloperidol (4-(4-[4-chlorophenyl]-4-hydroxy-1-piperidinyl)-1-(4-fluorophenyl)-1-butanone, MW 375.9) was obtained from Sigma Aldrich (St. Louis, Mo., USA).

Preparation of Compounds:

Compound 1 was solubilized in saline. Haloperidol was solubilized in water+acetic acid, and pH normalized to 5.0 with NaOH. All compounds were administered in solution in a volume of 0.1 ml/10 g body weights.

Experimental Procedure:

Startle response and PPI were measured using startle chambers from Hamilton Kinder (Poway, Calif., USA). Each chamber contained a plexiglas rectangle with an adjustable ceiling housed in a ventilated sound-attenuated cubicle. The ceiling was adjusted on an individual (animal by animal) basis to allow for adequate headroom but no rears or extensive locomotion. The chamber was placed over an anchor plate attached to a piezoelectric disk to transduce startle responses to a computer. A loudspeaker located in each chamber delivered the background noise (65 dB) and the acoustic stimuli. A constant white noise was maintained in the experimental room for the duration of the experiment by a white noise generator (Radio Shack, USA). Each session is initiated with a 5-minute acclimation period followed by four successive 120 dB, 40 ms trials. These trials were not included in the main analysis, but were referred to as baseline responses. Animals were then presented with 5 different trial types. Startle pulse (120 dB, 40 ms), or prepulse stimulus of one of three sound levels (70, 75, or 80 dB) for 20 ms, followed 100 ms later by an acoustic startle (120 dB) for 40 ms, or no stimulus at all. A total of 12 trials under each condition were delivered in a random sequence and all trials were separated by a variable inter-trial interval of 5-25 s. Finally, this sequence ended with the presentation of four 120 dB, 40 ms sound bursts (not included in the main analyses, but included in the baseline or habituation analyses). The animals were injected with the test compounds 30 minutes before the start of the trials. For co-administration studies, the α7 agonist was administered 10 minutes before haloperidol. Trials were initiated 30 minutes after the second injection. In the startle alone trials, the amount of PPI was calculated as a percentage score for each acoustic prepulse trial type using (typically) the formula:

[(startle response for prepulse+pulse)/(startle response for pulse alone)]*100.

Statistics:

Data were first analyzed using a two-way repeated measures analysis of variance (ANOVA) with two independent factors. If there was a significant interaction of both factors, subsequent post hoc one-way ANOVA were performed using each treatment combination as an independent group. All post hoc significance was determined using Dunnett's multiple comparison test (p<0.05 was regarded as significance).

Results:

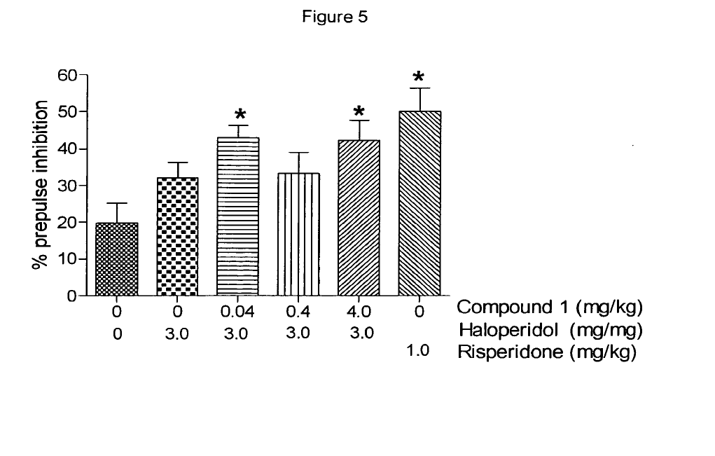

As shown in FIG. 5, no attenuation of haloperidol was noted. In fact, a significant potentiation of effects was observed in presence of Compound 1. The level of efficacy seen with the combination was equivalent to the efficacy seen with a high dose of the atypical antipsychotic risperidone, a degree of efficacy that is at or near maximal for the model.

### Example 6

### Evaluation of Compound 2, an α7 Neuronal Nicotinic Receptor Compound, in DBA Mice

The PPI model was used to investigate the effect of another selective α7 nicotinic receptor agonist, Compound 2.

Animals:

Male DBA/2J mice (Jackson Labs AX9 facility, Bar Harbor, Me., USA) at age 6-8 weeks old were used. They were housed under standard facility conditions in groups of eight on a 12 h light/dark cycle (lights on at 0600 h) with ad libitum access to food and water.

Chemicals:

Compound 2 (lot #1115256), 2-(6-phenylpyridazin-3-yl)octahydropyrrolo[3,4-c]pyrrole, MW 380.32 was prepared at Abbott Laboratories.

Preparation of Compounds:

Compound 2 was solubilized in saline. Compound was administered in solution in a volume of 0.1 ml/10 g body weights.

Experimental Procedure:

Startle response and PPI were measured using startle chambers from Hamilton Kinder (Poway, Calif., USA). Each chamber contained a plexiglas rectangle with an adjustable ceiling housed in a ventilated sound-attenuated cubicle. The ceiling was adjusted on an individual (animal by animal) basis to allow for adequate headroom but no rears or extensive locomotion. The chamber was placed over an anchor plate attached to a piezoelectric disk to transduce startle responses to a computer. A loudspeaker located in each chamber delivered the background noise (65 dB) and the acoustic stimuli. A constant white noise was maintained in the experimental room for the duration of the experiment by a white noise generator (Radio Shack, USA). Each session was initiated with a 5-minute acclimation period followed by four successive 120 dB, 40 ms trials. These trials were not included in the main analysis, but are referred to as baseline responses. Animals were then presented with 5 different trial types. Startle pulse (120 dB, 40 ms), or prepulse stimulus of one of three sound levels (70, 75, or 80 dB) for 20 ms, followed 100 ms later by an acoustic startle (120 dB) for 40 ms, or no stimulus at all. A total of 12 trials under each condition were delivered in a random sequence and all trials were separated by a variable inter-trial interval of 5-25 s. Finally, this sequence ended with the presentation of four 120 dB, 40 ms sound bursts (not included in the main analyses, but included in the baseline or habituation analyses). The animals were injected with the test compounds 30 minutes before the start of the trials. In the startle alone trials, the amount of PPI was calculated as a percentage score for each acoustic prepulse trial type using (typically) the formula:

[(startle response for prepulse+pulse)/(startle response for pulse alone)]*100.

Statistics:

Data were first analyzed using a two-way repeated measures analysis of variance (ANOVA) with two independent factors. If there was a significant interaction of both factors, subsequent post hoc one-way ANOVA were performed using each treatment combination as an independent group. All post hoc significance was determined using Dunnett's multiple comparison test (p<0.05 was regarded as significance).

Results:

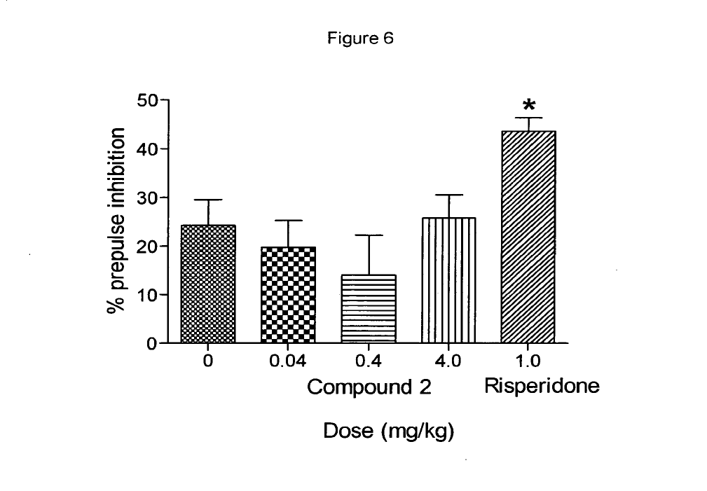

As shown in FIG. 6, Compound 2 alone showed no effect at 0.04-4.0 mg/kg.

### Example 7

### Compound 2, an α7 Agonist, Potentiated the Antipsychotic Effect of Risperidone

The PPI model was used to investigate the effect of another selective α7 nicotinic receptor agonist, Compound 2 (0.04-4.0 mg/kg) on a subefficacious dose of risperidone.

Animals:

Male DBA/2J mice (Jackson Labs AX9 facility, Bar Harbor, Me., USA) at age 6-8 weeks old were used. They were housed under standard facility conditions in groups of eight on a 12 h light/dark cycle (lights on at 0600 h) with ad libitum access to food and water.

Chemicals:

Compound 2 (lot #1115256), MW 380.32 was prepared at Abbott Laboratories; risperidone (3-[2-[4-(6-fluoro-1,2-benzisoxazol-3-yl)-1-piperidinyl]ethyl]-6,7,8,9-tetrahydro-2-methyl-4H-pyridol[1,2-a]pyrimidin-4-one, MW 410.5) was obtained from ICN Biomedicals Inc. (Aurora, Ohio, USA)

Preparation of Compounds:

Compound 2 was solubilized in saline. Risperidone was solubilized in water+acetic acid, and pH normalized to 5.5 with NaOH. All compounds were administered in solution in a volume of 0.1 ml/10 g body weights.

Experimental Procedure:

Startle response and PPI were measured using startle chambers from Hamilton Kinder (Poway, Calif., USA). Each chamber contained a plexiglas rectangle with an adjustable ceiling housed in a ventilated sound-attenuated cubicle. The ceiling was adjusted on an individual (animal by animal) basis to allow for adequate headroom but no rears or extensive locomotion. The chamber was placed over an anchor plate attached to a piezoelectric disk to transduce startle responses to a computer. A loudspeaker located in each chamber delivered the background noise (65 dB) and the acoustic stimuli. A constant white noise was maintained in the experimental room for the duration of the experiment by a white noise generator (Radio Shack, USA). Each session was initiated with a 5-minute acclimation period followed by four successive 120 dB, 40 ms trials. These trials were not included in the main analysis, but are referred to as baseline responses. Animals were then presented with 5 different trial types. Startle pulse (120 dB, 40 ms), or prepulse stimulus of one of three sound levels (70, 75, or 80 dB) for 20 ms, followed 100 ms later by an acoustic startle (120 dB) for 40 ms, or no stimulus at all. A total of 12 trials under each condition were delivered in a random sequence and all trials were separated by a variable inter-trial interval of 5-25 s. Finally, this sequence ended with the presentation of four 120 dB, 40 ms sound bursts (not included in the main analyses, but included in the baseline or habituation analyses). The animals were injected with the test compounds 30 minutes before the start of the trials. For co-administration studies, the α7 agonist was administered 10 minutes before risperidone. Trials were initiated 30 minutes after the second injection. In the startle alone trials, the amount of PPI was calculated as a percentage score for each acoustic prepulse trial type using (typically) the formula:

[(startle response for prepulse+pulse)/(startle response for pulse alone)]*100.

Statistics:

Data were first analyzed using a two-way repeated measures analysis of variance (ANOVA) with two independent factors. If there was a significant interaction of both factors, subsequent post hoc one-way ANOVA were performed using each treatment combination as an independent group. All post hoc significance was determined using Dunnett's multiple comparison test (p<0.05 was regarded as significance).

Results:

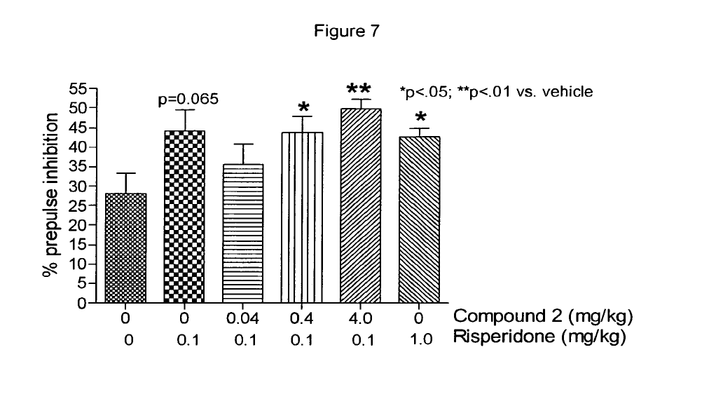

As shown in FIG. 7, risperidone (0.1 mg/kg) was found to be more efficacious in the presence of Compound 2 and attained maximal efficacy (comparable to that observed by 1.0 mg/kg risperidone) in combination with 0.4-4.0 mg/kg Compound 2. This demonstrates that a dose that is normally weakly efficacious can be made to exhibit robust efficacy upon α7 receptor activation by another selective agonist.

### Example 8

### Evaluation of Compound 3, an α7 Neuronal Nicotinic Receptor Compound, in DBA Mice

The PPI model was used to investigate the effect of another selective al nicotinic receptor agonist, Compound 3.

Animals:

Male DBA/2J mice (Jackson Labs AX9 facility, Bar Harbor, Me., USA) at age 6-8 weeks old were used. They were housed under standard facility conditions in groups of eight on a 12 h light/dark cycle (lights on at 0600 h) with ad libitum access to food and water.

Chemicals:

Compound 3 (lot #1163769), N-(3R)-1-azabicyclo[2,2,2]oct-3-yl-4-chlorobenzamide fumarate, MW 402.45 was synthesized at Abbott Laboratories. Alternatively, Compound 3 can be obtained from Tocris (Ellisville, Mo., USA).

Preparation of Compounds:

Compound 3 was solubilized in saline. Compound 3 was administered in solution in a volume of 0.1 ml/10 g body weights.

Experimental Procedure:

Startle response and PPI were measured using startle chambers from Hamilton Kinder (Poway, Calif., USA). Each chamber contained a plexiglas rectangle with an adjustable ceiling housed in a ventilated sound-attenuated cubicle. The ceiling was adjusted on an individual (animal by animal) basis to allow for adequate headroom but no rears or extensive locomotion. The chamber was placed over an anchor plate attached to a piezoelectric disk to transduce startle responses to a computer. A loudspeaker located in each chamber delivered the background noise (65 dB) and the acoustic stimuli. A constant white noise was maintained in the experimental room for the duration of the experiment by a white noise generator (Radio Shack, USA). Each session was initiated with a 5-minute acclimation period followed by four successive 120 dB, 40 ms trials. These trials were not included in the main analysis, but are referred to as baseline responses. Animals were then presented with 5 different trial types. Startle pulse (120 dB, 40 ms), or prepulse stimulus of one of three sound levels (70, 75, or 80 dB) for 20 ms, followed 100 ms later by an acoustic startle (120 dB) for 40 ms, or no stimulus at all. A total of 12 trials under each condition were delivered in a random sequence and all trials were separated by a variable inter-trial interval of 5-25 s. Finally, this sequence ended with the presentation of four 120 dB, 40 ms sound bursts (not included in the main analyses, but included in the baseline or habituation analyses). The animals were injected with the test compounds 30 minutes before the start of the trials. In the startle alone trials, the amount of PPI was calculated as a percentage score for each acoustic prepulse trial type using (typically) the formula:

[(startle response for prepulse+pulse)/(startle response for pulse alone)]*100.

Statistics:

Data were first analyzed using a two-way repeated measures analysis of variance (ANOVA) with two independent factors. If there was a significant interaction of both factors, subsequent post hoc one-way ANOVA were performed using each treatment combination as an independent group. All post hoc significance was determined using Dunnett's multiple comparison test (p<0.05 was regarded as significance).

Results:

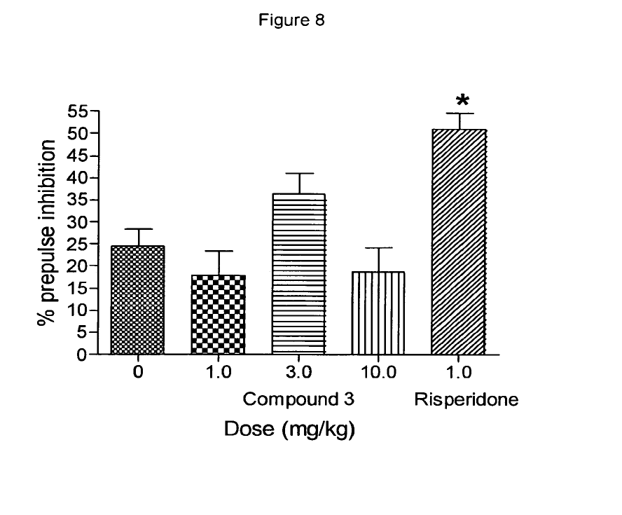

As shown in FIG. 8, Compound 3 alone showed no effect at 1.0-10.0 mg/kg.

### Example 9

### α7 Agonists Potentiated Antipsychotic Effect of Risperidone

The PPI model was used to investigate the effect of a selective α7 nicotinic receptor agonist, Compound 3 (1.0-10.0 mg/kg), on a subefficacious dose of risperidone.

Animals:

Male DBA/2J mice (Jackson Labs AX9 facility, Bar Harbor, Me., USA) at age 6-8 weeks old were used. They were housed under standard facility conditions in groups of eight on a 12 h light/dark cycle (lights on at 0600 h) with ad libitum access to food and water.

Chemicals:

Compound 3, N-(3R)-1-azabicyclo[2,2,2]oct-3-yl-4-chlorobenzamide fumarate, MW 402.45, was prepared at Abbott Laboratories; risperidone (3-[2-[4-(6-fluoro-1,2-benzisoxazol-3-yl)-1-piperidinyl]ethyl]-6,7,8,9-tetrahydro-2-methyl-4H-pyridol[1,2-a]pyrimidin-4-one, MW 410.5) was obtained from ICN Biomedicals Inc. (Aurora, Ohio, USA).

Preparation of Compounds:

Compound 3 was solubilized in saline. Risperidone was solubilized in water+acetic acid, and pH normalized to 5.5 with NaOH. All compounds were administered in solution in a volume of 0.1 ml/10 g body weights.

Experimental Procedure:

Startle response and PPI were measured using startle chambers from Hamilton Kinder (Poway, Calif., USA). Each chamber contained a plexiglas rectangle with an adjustable ceiling housed in a ventilated sound-attenuated cubicle. The ceiling was adjusted on an individual (animal by animal) basis to allow for adequate headroom but no rears or extensive locomotion. The chamber was placed over an anchor plate attached to a piezoelectric disk to transduce startle responses to a computer. A loudspeaker located in each chamber delivered the background noise (65 dB) and the acoustic stimuli. A constant white noise was maintained in the experimental room for the duration of the experiment by a white noise generator (Radio Shack, USA). Each session was initiated with a 5-minute acclimation period followed by four successive 120 dB, 40 ms trials. These trials were not included in the main analysis, but were referred to as baseline responses. Animals were then presented with 5 different trial types. Startle pulse (120 dB, 40 ms), or prepulse stimulus of one of three sound levels (70, 75, or 80 dB) for 20 ms, followed 100 ms later by an acoustic startle (120 dB) for 40 ms, or no stimulus at all. A total of 12 trials under each condition were delivered in a random sequence and all trials were separated by a variable inter-trial interval of 5-25 s. Finally, this sequence ended with the presentation of four 120 dB, 40 ms sound bursts (not included in the main analyses, but included in the baseline or habituation analyses). The animals were injected with the test compounds 30 minutes before the start of the trials. For co-administration studies, the α7 agonist was administered 10 minutes before risperidone. Trials were initiated 30 minutes after the second injection. In the startle alone trials, the amount of PPI was calculated as a percentage score for each acoustic prepulse trial type using (typically) the formula:

[(startle response for prepulse+pulse)/(startle response for pulse alone)]*100.

Statistics:

Data were first analyzed using a two-way repeated measures analysis of variance (ANOVA) with two independent factors. If there was a significant interaction of both factors, subsequent post hoc one-way ANOVA were performed using each treatment combination as an independent group. All post hoc significance was determined using Dunnett's multiple comparison test (p<0.05 was regarded as significance).

Results:

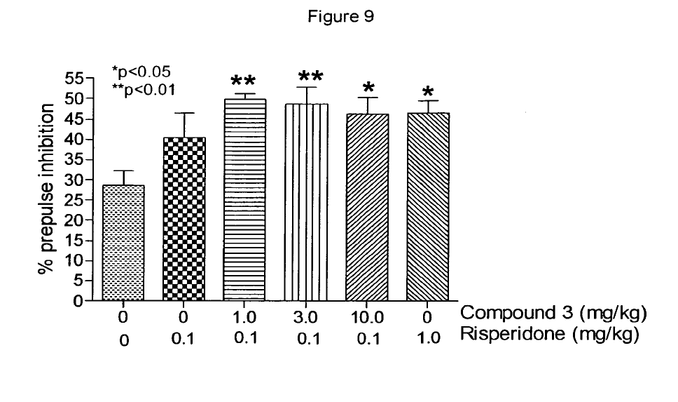

As shown in FIG. 9, risperidone (0.1 mg/kg) was found to be more efficacious in the presence of Compound 3 and attained maximal efficacy (comparable to that observed by 1.0 mg/kg risperidone) in combination with 1.0-10.0 mg/kg of Compound 3. This demonstrated that a dose that is normally weakly efficacious can be made to exhibit robust efficacy upon α7 receptor activation by another selective agonist.

### Example 10

### α7 Agonists Do Not Exacerbate the Cataleptic Effect of Haloperidol

One of the adverse effects antipsychotic mediations is extrapyramidal movement disorder syndrome attributed to blockade of the dopamine D2 receptors. Extrapyramidal movement disorder can be predicted by the cataleptic response elicited by an antipsychotic in a rodent. To assess whether Compound 1 alone evoked cataleptic responses or interfered with the catalpetic effect of the antipsychotic, the following set of studies were conducted.

Animals:

Male Sprague Dawley rats (CRL: CD (SD), Charles River Laboratories, Omaha, Nebr., USA) weighing 300-325 g were used for the experiment. They were housed under standard conditions in groups of 4 rats on a 12 h light/dark cycle (lights on at 0600 h) with ad libitum access to food and water.

Chemicals:

Compound 1 (lot #1278527), MW 402.32 was prepared at Abbott Laboratories; haloperidol (4-(4-[4-chlorophenyl]-4-hydroxy-1-piperidinyl)-1-(4-fluorophenyl)-1-butanone, MW 375.9) was obtained from Sigma Aldrich (St. Louis, Mo., USA).

Preparation of Compounds:

Compound 1 was solubilized in saline. Haloperidol was solubilized in water+acetic acid, and pH normalized to 5.0 with NaOH. All compounds were administered in solution in a volume of 1.0 ml/kg body weight.

Experimental Procedure:

Rats were handled and habituated to the testing room before starting. On test day rats were transferred into individual cages and left undisturbed for at least one hour. All compound were dosed at 1.0 ml/kg i.p. In, the case of co-treatment, the α7 agonist was administered 10 minutes prior to the haloperidol. Rats were tested at 60, 120, 180, and 240 minutes post-injection for cataleptic responses and returned to cages in-between test sessions. The degree of catalepsy was measured by gently placing both forepaws over a metal bar (1.1 cm. diameter suspended 8 cm. above the table top). The time in seconds until the rat took both paws off the bar was recorded, with a maximum cut-off of 300 seconds. The total duration of catalepsy in the different time points was used for analysis. At least 5 trials were attempted on each rat with 5 seconds used as a low-end cut-off for catalepsy (time scored as zero). For catalepsy times between 5-15 seconds, the highest time of 5 trials was recorded. Alternately, any catalepsy trial time that was greater than 15 seconds (up to 300 seconds) was recorded.

Statistics:

Data were first analyzed using a two-way repeated measures analysis of variance (ANOVA) with two independent factors. If there was a significant interaction of both factors, subsequent post hoc one-way ANOVAs were performed using each treatment combination as an independent group. All post hoc significance was determined using a Student's t-test. (p<0.05 was regarded as significant).

Results:

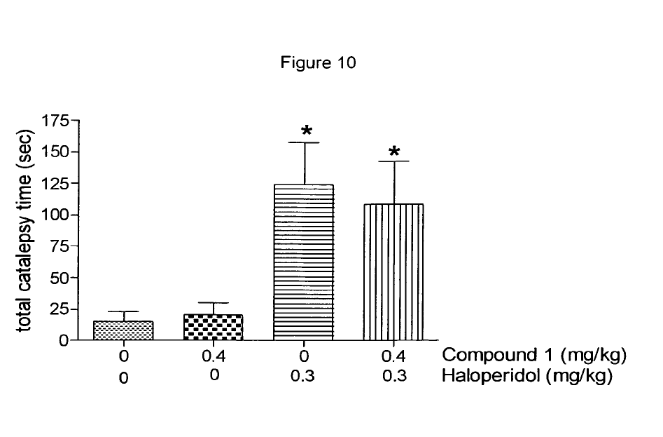

As shown in FIG. 10, Compound 1 (0.4 mg/kg i.p.) did not alter cataleptic behavior of haloperidol (0.3 mg/kg i.p.), when administered in combination. This study demonstrates that an α7 agonist does not have the potential to alter the side effect profile of the antipsychotic drug. However, since efficacy of the antipychotic drug is improved at comparable doses, the net effect would be an overall improvement in therapeutic window in combination with α7 neuronal nicotinic receptor ligand.

### Example 11

### α7 Agonist Compound 2 Does Not Exacerbate the Cataleptic Effect of Risperidone

Animals:

Male Sprague Dawley rats (CRL: CD (SD), Charles River Laboratories, Omaha, Nebr., USA) weighing 300-325 g were used for the experiment. They were housed under standard conditions in groups of 4 rats on a 12 h light/dark cycle (lights on at 0600 h) with ad libitum access to food and water.

Chemicals:

Compound 2 (lot #1115256), MW 380.32 was prepared at Abbott Laboratories; risperidone (3-[2-[4-(6-fluoro-1,2-benzisoxazol-3-yl)-1-piperidinyl]ethyl]-6,7,8,9-tetrahydro-2-methyl-4H-pyridol[1,2-a]pyrimidin-4-one, MW 410.5) was obtained from ICN Biomedicals Inc. (Aurora, Ohio, USA).

Preparation of Compounds:

Compound 2 was solubilized in saline. Risperidone was solubilized in water+acetic acid, and pH normalized to 5.5 with NaOH. All compounds were administered in solution in a volume of 1.0 ml/kg body weight.

Experimental Procedure:

Rats were handled and habituated to the testing room before starting. On test day rats were transferred into individual cages and left undisturbed for at least one hour. All compounds were dosed at 1.0 ml/kg i.p. In the case of co-treatment, the α7 agonist was administered 10 minutes prior to the risperidone. Rats were tested at 60, 120, 180, and 240 minutes post-injection for cataleptic responses and returned to cages in-between test sessions. The degree of catalepsy was measured by gently placing both forepaws over a metal bar (1.1 cm. diameter suspended 8 cm. above the table top). The time in seconds until the rat took both paws off the bar was recorded, with a maximum cut-off of 300 seconds. The total duration of catalepsy in the different time points was used for analysis. At least 5 trials were attempted on each rat with 5 seconds used as a low-end cut-off for catalepsy (time scored as zero). For catalepsy times between 5-15 seconds, the highest time of 5 trials was recorded. Alternately, any catalepsy trial time that was greater than 15 seconds (up to 300 seconds) was recorded.

Statistics:

Data were first analyzed using a two-way repeated measures analysis of variance (ANOVA) with two independent factors. If there was a significant interaction of both factors, subsequent post hoc one-way ANOVAs were performed using each treatment combination as an independent group. All post hoc significance was determined using a Student's t-test. (p<0.05 was regarded as significant).

Results:

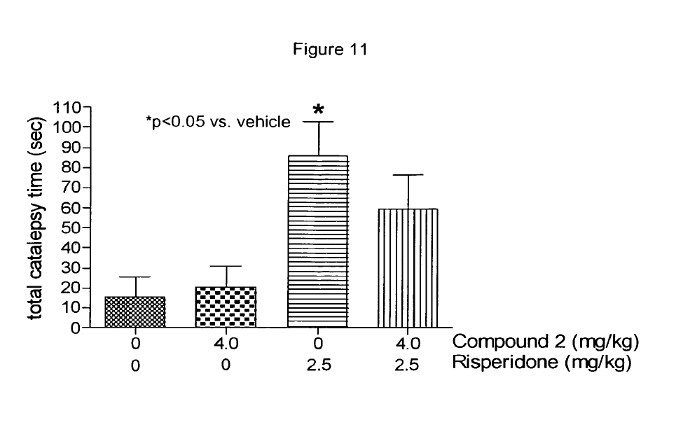

As shown in FIG. 11, Compound 2 (4.0 mg/kg i.p.) did not significantly alter cataleptic behavior of risperidone (2.5 mg/kg i.p.), when administered in combination. This study demonstrates that another α7 agonist does not have the potential to alter the side effect profile of the antipsychotic drug. However, since efficacy of the antipychotic drug is improved at comparable doses, the net effect would be an overall improvement in therapeutic window in combination with α7 neuronal nicotinic receptor ligand.

### Example 12

### α7 Agonist, Compound 3, Does Not Exacerbate the Cataleptic Effect of Risperidone

Animals:

Male Sprague Dawley rats (CRL: CD (SD), Charles River Laboratories, Omaha, Ne) weighing 300-325 g were used for the experiment. They were housed under standard conditions in groups of 4 rats on a 12 h light/dark cycle (lights on at 0600 h) with ad libitum access to food and water.

Chemicals:

Compound 3, MW 402.45, was prepared at Abbott Laboratories; risperidone (3-[2-[4-(6-fluoro-1,2-benzisoxazol-3-yl)-1-piperidinyl]ethyl]-6,7,8,9-tetrahydro-2-methyl-4H-pyridol[1,2-a]pyrimidin-4-one, MW 410.5) was obtained from ICN Biomedicals Inc. (Aurora, Ohio, USA).

Preparation of Compounds:

Compound 3 was solubilized in saline. Risperidone was solubilized in water+acetic acid, and pH normalized to 5.5 with NaOH. All compounds were administered in solution in a volume of 1.0 ml/kg body weight.

Experimental Procedure:

Rats were handled and habituated to the testing room before starting. On test day rats were transferred into individual cages and left undisturbed for at least one hour. All compounds were dosed at 1.0 ml/kg i.p. In the case of co-treatment, the α7 agonist was administered 10 minutes prior to the risperidone. Rats were tested at 60, 120, 180, and 240 minutes post-injection for cataleptic responses and returned to cages in-between test sessions. The degree of catalepsy was measured by gently placing both forepaws over a metal bar (1.1 cm. diameter suspended 8 cm. above the table top). The time in seconds until the rat took both paws off the bar was recorded, with a maximum cut-off of 300 seconds. The total duration of catalepsy in the different time points was used for analysis. At least 5 trials were attempted on each rat with 5 seconds used as a low-end cut-off for catalepsy (time scored as zero). For catalepsy times between 5-15 seconds, the highest time of 5 trials was recorded. Alternately, any catalepsy trial time that was greater than 15 seconds (up to 300 seconds) was recorded.

Statistics:

Data were first analyzed using a two-way repeated measures analysis of variance (ANOVA) with two independent factors. If there was a significant interaction of both factors, subsequent post hoc one-way ANOVAs were performed using each treatment combination as an independent group. All post hoc significance was determined using a Student's t-test. (p<0.05 was regarded as significant).

Results:

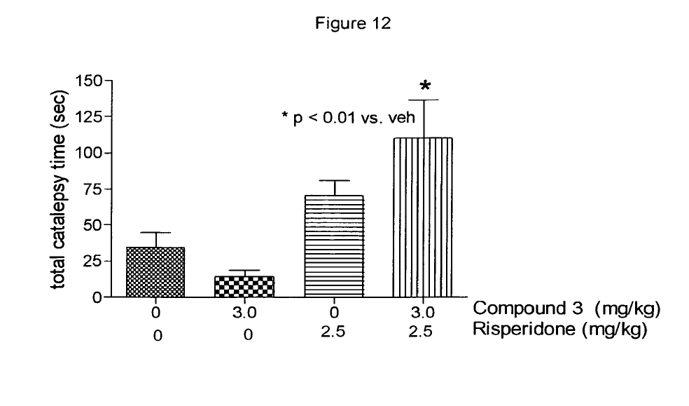

As shown in FIG. 12, Compound 3 (3.0 mg/kg i.p.) did not significantly alter cataleptic behavior of risperidone (2.5 mg/kg i.p.), when administered in combination. This study demonstrates that another α7 agonist does not have the potential to alter the side effect profile of the antipsychotic drug. However, since efficacy of the antipychotic drug is improved at comparable doses, the net effect would be an overall improvement in therapeutic window in combination with α7 neuronal nicotinic receptor ligand.

The compositions, methods, and articles of manufacture have been described with reference to various specific embodiments and techniques. The examples described herein illustrate but do not limit the scope of the invention as defined in the appended claims and equivalents thereof.

What is claimed is:

1. A composition, comprising:
(i) an antipsychotic; and
(ii) an neuronal nicotinic subtype α7 receptor ligand;
in admixture with at least one pharmaceutically acceptable excipient.

2. The composition of claim 1, wherein the neuronal nicotinic receptor α7 receptor ligand demonstrates a ratio of the Kᵢ value that as measured by [³H]-cytisine binding assay (Kᵢ Cyt) to the Kᵢ value as measured by MLA binding assay (Kᵢ MLA) in a formula D=Kᵢ Cyt/Kᵢ MLA such that D is greater than a value of 50.

3. The composition of claim 1, wherein the neuronal nicotinic subtype α7 receptor ligand is a neuronal nicotinic subtype α7 agonist, neuronal nicotinic subtype α7 partial agonist, or neuronal nicotinic subtype α7 allosteric modulator.

4. The composition of claim 1, wherein the neuronal nicotinic subtype α7 receptor ligand is selected from the group consisting of diazabicycloalkane derivatives, spirocyclic quinuclidinic ether derivatives, bicycloheterocycle substituted quinuclidine derivatives, 3-quinuclidinyl amino-substituted biaryl derivatives, 3-quinuclidinyl heteroatom-bridged biaryl derivatives, and amino-substituted tricyclic derivatives.

5. The composition of claim 1, wherein the neuronal nicontinic subtype α7 receptor ligand has the formula:
Z—Ar¹—Ar² (I)
or a pharmaceutically acceptable salt, ester, amide, or prodrug thereof, wherein:
Z is a diazabicyclic amine of the formula: 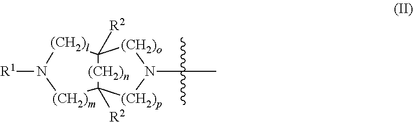
Ar¹ is a 5- or 6-membered aromatic ring of the formula (a) or (b): 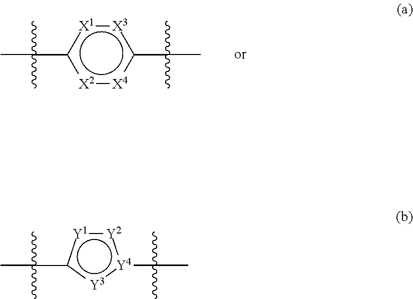
Ar² is selected from the group consisting of an unsubstituted or substituted 5- or 6-membered heteroaryl ring; unsubstituted or substituted bicyclic heteroaryl ring; 3,4-(methylenedioxy)phenyl; carbazolyl; tetrahydrocarbazolyl; naphthyl; and phenyl; wherein Ar² is substituted with 0, 1, 2, or 3 substituents selected from the group consisting of alkenyl, alkoxy, alkoxyalkoxy, alkoxyalkyl, alkoxycarbonyl, alkoxysulfonyl, alkyl, alkylcarbonyl, arylcarbonyl, alkylcarbonyloxy, alkylsulfonyl, alkylthio, alkynyl, carboxy, cyano, formyl, haloalkoxy, haloalkyl, halogen, hydroxy, hydroxyalkyl, mercapto, nitro, —NR^{A}R^{B}, (NR^{A}R^{B})alkyl, (NR^{A}R^{B})carbonyl, (NR^{A}R^{B})sulfonyl, and phenyl; provided that when Y¹ is O or S, Y² is N, Y³ is —CR³ and R³ is hydrogen, and Y⁴ is C, then Ar² is not 5-tetrazolyl;
X¹, X², X³, and X⁴ are each independently selected from the group consisting of N and —CR³, provided that R³ is not hydrogen at least in one occurrence when X¹, X², X³, and X⁴ are all —CR³;
Y¹, Y², and Y³ are each independently selected from the group consisting of N, O, S, and —CR³;
Y⁴ is selected from the group consisting of C and N, provided that when Y⁴ is C at least one of Y¹, Y², and Y³, is other than —CR³;
l, m, n, o, and p are each independently selected from the group consisting of 0, 1, or 2, provided that the sum total of l, m, n, o, and p is 3, 4, or 5, and further provided that the sum of l and o is at least 1 and the sum of m and p is at least 1;
R¹ is selected from the group consisting of hydrogen, alkenyl, alkyl alkoxycarbonyl, arylalkyl, and heteroarylalkyl;
R² at each occurrence is independently selected from the group consisting of hydrogen, alkoxycarbonyl, and alkyl;
R³ at each occurrence is independently selected from the group consisting of hydrogen and alkyl;
R^{A} and R^{B} are each independently selected from the group consisting of hydrogen, alkyl, alkylcarbonyl, alkylsulfonyl, arylcarbonyl, formyl and (NR^{C}R^{D})sulfonyl; and
R^{C} and R^{D} are each independently selected from the group consisting of hydrogen and alkyl.

6. The composition of claim 1, wherein the neuronal nicontinic subtype α7 receptor ligand has the formula: 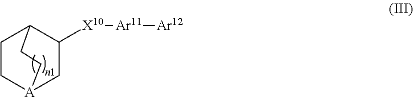
or a pharmaceutically acceptable salt, ester, amide, or prodrug thereof, wherein:
n1 is 0, 1, or 2;
A is N or N⁺—O⁻;
X¹⁰ is selected from the group consisting of O, S, and —N(R¹¹)—;
Ar¹¹ is a 6-membered aromatic ring containing 0, 1, 2, 3, or 4 nitrogen atoms, wherein Ar¹¹ is substituted with 0, 1, 2, 3, or 4 alkyl groups;
Ar¹² is a group of the formula: 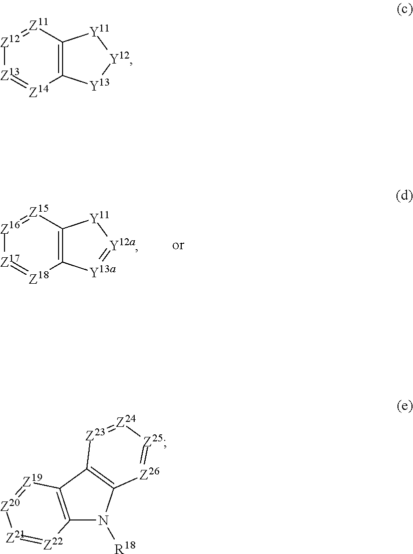
Z¹¹, Z¹², Z¹³, and Z¹⁴ are independently selected from the group consisting of C and —C(R^{3b}); provided that zero or one of Z¹¹, Z¹², Z¹³, and Z¹⁴ is C;
Z¹⁵, Z¹⁶, Z¹⁷, and Z¹⁸ are independently selected from the group consisting of C and —C(R^{3b}); provided that zero or one of Z¹⁵, Z¹⁶, Z¹⁷, and Z¹⁸ is C;
Z¹⁹, Z²⁰, Z²¹, Z²², Z²³, Z²⁴, Z²⁵, and Z²⁶ are independently selected from the group consisting of C and —C(R^{3c}); provided that one of Z¹⁹, Z²⁰, Z²¹, Z²², Z²³, Z²⁴, Z²⁵, and Z²⁶ is C and the group of formula (e) is attached to Ar¹ through the C atom;
Y¹¹ at each occurrence is independently selected from the group consisting of O, S, —N(R₁₂), —C(R¹³), and —C(R¹³)(R^{13a});
Y¹² is selected from the group consisting of —N(R¹²), C(═O), —C(R¹³), and —C(R¹³)(R^{13a});
Y¹³ is selected from the group consisting of —N(R¹²), —C(R¹³), and —C(R¹³)(R^{13a}); provided that zero or one of Y¹¹, Y¹², and Y¹³ is —C(R¹³) in a group of formula (c);
wherein when one of Y¹¹, Y¹², and Y¹³ is —C(R¹³) in a group of formula (c), then Z¹¹, Z¹², Z¹³, and Z¹⁴ are each —C(R^{13b}) and the group of formula (c) is attached to Ar¹¹ through the C atom of —C(R¹³) of Y¹¹, Y¹², or Y¹³; and also when one of Z¹¹, Z¹², Z¹³, and Z¹⁴ is C, then Y¹¹, Y¹², and Y¹³ are other than —C(R¹³) and the group of formula (c) is attached to Ar¹¹ through the C atom of Z¹¹, Z¹², Z¹³, or Z¹⁴;
Y^{12a} and Y^{13a} are independently selected from the group consisting of N, C and —C(R^{13a}); provided that when Y¹¹ is —C(R¹³) in a group of formula (d), Y^{12a} and Y^{3a} are selected from the group consisting of N and —C(R¹³), and when one of Y^{12a} and Y^{13a} is C, then Y¹¹ in a group of formula (d) is O, S, —N(R¹²), or —C(R¹³)(R^{13a});
wherein when one of Z¹⁵, Z¹⁶, Z¹⁷, and Z¹⁸ is C, then Y¹¹ in a group of formula (d) is selected from the group consisting of O, S, —N(R₁₂), and —C(R¹³)(R^{13a}); Y^{12a} and Y^{13a} are each independently selected from the group consisting of N and —C(R^{13a}); and the group of formula (b) is attached to Ar¹¹ through the C of Z¹⁵, Z¹⁶, Z¹⁷ or Z¹⁸; and also wherein when Y¹¹ in a group of formula (d) is —C(R¹³) or one of Y^{12a} and Y^{13a} is C, then Z¹⁵, Z¹⁶, Z¹⁷, and Z¹⁸ are each —C(R^{13b}) and the group of formula (d) is attached to Ar¹¹ through the C atom of —C(R¹³) of Y¹¹ in the group of formula (d) or through the C atom of Y^{12a} or Y^{13a};
R¹¹ and R¹² at each occurrence are each independently selected from the group consisting of hydrogen and alkyl;
R¹³ and R^{13a} at each occurrence are each independently selected from the group consisting of hydrogen, halogen, alkyl, aryl, —OR, —NR¹⁵R¹⁶, -alkyl-OR¹⁴, and -alkyl-NR¹⁵R¹⁶;
R^{13b} and R^{13c} at each occurrence are each independently selected from the group consisting of hydrogen, halogen, alkyl, aryl, —OR¹⁴, —NR¹⁵R¹⁶, -alkyl-OR¹⁴, -alkyl-NR¹⁵R¹⁶, and —SCN;
R¹⁴ is selected from the group consisting of hydrogen, alkyl, aryl, alkylcarbonyl, and arylcarbonyl;
R¹⁵ and R¹⁶ at each occurrence are each independently selected from the group consisting of hydrogen, alkyl, aryl, alkylcarbonyl, alkoxycarbonyl, aryloxycarbonyl, and arylcarbonyl, provided that at least one of R¹⁵ and R¹⁶ is hydrogen or alkyl; and
R¹⁸ is selected from the group consisting of hydrogen and alkyl.

7. The composition of claim 1, wherein the neuronal nicotinic subtype α7 receptor ligand is selected from the group consisting of:
5-(6-[(3R)-1-azabicyclo[2.2.2]oct-3-yloxy]pyridazin-3-yl)-1H-indole;
2-(6-phenylpyridazine-3-yl)octahydropyrrolo[3,4-c]pyrrole;
5-[5-{(1R,5R)-6-methyl-3,6-diaza-bicyclo[3.2.0]hept-3-yl}-pyridin-2-yl]-1H-indole; and
5-[6-(cis-5-methyl-hexahydro-pyrrolo[3,4-c]pyrrol-2-yl)-pyridazin-3-yl-1H-indole.

8. The composition of claim 1, wherein the neuronal nicotinic subtype α7 receptor ligand is N-[(3R)-1-azabicyclo[2.2.2]oct-3-yl]-4-chlorobenzamide (PNU-282987), MEM-3454, AR R-1119, AZD0328, WB-56203, SSR-180711A, GTS21, OH-GTS-21, TC-5619, or varenicline.

9. The composition of claim 1, wherein the atypical antipsychotic is present in a sub efficacious amount for treating a psychotic condition.

10. The composition of claim 1, wherein the antipsychotic is selected from the group consisting of haloperidol, risperidone, olanzapine, clozapine, quetiapine, ziprasidone, aripiprazole, sertindole, zotepine, and perospirone.

11. A method for use in treating or preventing a psychotic condition in a patient, comprising:
(i) administering an amount of antipsychotic to the patient; and
(ii) administering an amount of neuronal nicotinic receptor subtype α7 receptor ligand to the patient;
wherein the amounts of (i) and (ii) together are effective in treating a psychotic or affective disorder.

12. The method of claim 11, wherein the amount of (i) is a sub efficacious amount.

13. The method of claim 11, wherein the patient previously suffered extrapyramidal symptoms during treatment with an antipsychotic.

14. An article of manufacture, comprising:
(i) a first pharmaceutical dosage form comprising at least one antipsychotic;
(ii) a second pharmaceutical dosage form comprising at least one neuronal nicotinic acetylcholine subtype α7 receptor ligand;
wherein the article contains first and second pharmaceutical dosage forms.
